(19) United States
(12) Patent Application Publication
(10) Pub. No.: US 2026/0060794 A1
(43) Pub. Date: Mar. 5, 2026

(54) DEVICE AND METHOD FOR AN ENDO-BENTALL PROCEDURE

(71) Applicant: Bolton Medical, Inc., Sunrise, FL (US)

(72) Inventors: Casey Torrance, Snohomish, WA (US); Timothy Lostetter, Cooper City, FL (US); Panagiotis Peter Vassios, Plantation, FL (US); Alejandro Pinero, Miramar, FL (US); Samuel Arbefeuille, Hollywood, FL (US); Scott Lyle Rush, Sunrise, FL (US)

(21) Appl. No.: 19/316,596

(22) Filed: Sep. 2, 2025

Related U.S. Application Data
(60) Provisional application No. 63/688,984, filed on Aug. 30, 2024.

(51) Int. Cl. A61F 2/07; A61F 2/06; A61F 2/24

(52) U.S. Cl. CPC A61F 2/07; A61F 2/2418; A61F 2002/061; A61F 2002/067; A61F 2210/009; A61F 2230/0065; A61F 2230/0069; A61F 2250/001

(57) ABSTRACT

A medical device including a expandable graft and valve attachment and methods related to attaching the valve (with or without an elongated graft portion) to a fenestrated stent graft are disclosed. The device and methods disclosed herein are particularly suitable for Endo-Bentall procedures via a minimally invasive endovascular technique used to repair the aortic root and ascending aorta. An exemplary device has a larger diameter at a proximal end with fenestrations associated with tunnel lumens longitudinally aligned with the graft lumen. The proximal end of the main graft lumen receives a valve, wherein the graft lumen trifurcates in a retrograde manner parallel with the first tunnel and second tunnel. The first and second tunnel is configured to receive a first and second bridging stent.

### CROSS REFERENCE TO RELATED APPLICATIONS

This application claims the benefit of priority under 35 U.S.C. § 119 to U.S. Provisional Patent Application No. 63/688,984 filed Aug. 30, 2024.

### TECHNICAL FIELD

The disclosure is generally directed to the use of expandable grafts and methods for their delivery, and in particular, to stent-grafts for usage in a procedure to replace and repair the ascending aorta, aortic root, and/or aortic valve (e.g. Endo-Bentall procedures).

### BACKGROUND OF THE DISCLOSURE

Diseases of the aortic valve and ascending aorta present life-threatening conditions which require immediate treatment. Diseases of this type generally fall into three categories. The first are aortic root aneurysms, which generally involve the abnormal swelling or dilation of the aortic root. In these situations, the wall of the abnormally dilated blood vessel is typically weakened and susceptible to rupture. The second is aortic regurgitation, which is a condition wherein the aortic valve does not open and close properly, leading to fluid backflow in the aorta. Lastly, the third are aortic dissections which result in a tear in the aorta's inner lining, allowing blood to flow between the layers of the aortic wall, causing them to separate. This condition can lead to blood deprivation to the vital organs, leading to immediate death. For example, an acute type A dissection (ATAAD) is a type of aortic dissection which affects the aortic root and valve. A common way to treat the above-mentioned conditions is to place an endovascular stent graft such that the stent graft spans across and extends beyond the proximal and distal ends of the diseased portion of the vasculature. The stent graft is designed to reline the diseased vasculature, providing an alternate blood conduit that isolates the aneurysm or dissection from the high-pressure flow of blood, thereby reducing or eliminating the risk of rupture and/or providing a continuous, uninterrupted conduit for blood flow. The embodiments disclosed herein are particularly suited for treatment of aortic aneurysms and dissections that encompass or affect the aortic root and valve.

Traditional methods for treating aortic valve and ascending aorta diseases include highly invasive open-heart procedures, such as the Bentall procedure. During the Bentall procedure, the aortic valve, aortic root, and ascending aorta are replaced, and the coronary arteries are re-implanted into the graft. Although the Bentall procedure can be successful in some cases, the procedure presents multiple drawbacks and disadvantages. For instance, recovery times are often very long due to the traumatic nature of the procedure (i.e., cracking and opening of the sternum). Another consequence of this procedure is the high risk of infection after the procedure, which may last for weeks and even years after completion of the procedure. Furthermore, many patients are excluded from consideration in receiving the Bentall procedure. Since the procedure is highly traumatic, frail, elderly patients, patients with pre-existing conditions, such as diabetes or Chronic Obstructive Pulmonary Disease (COPD), and obese patients are all poor candidates, although they may experience diseases of the aorta and aortic valve at higher frequencies than those who may be considered suitable candidates.

Accordingly, minimally invasive endovascular repair using stent grafts is often preferred to avoid the risks associated with traditional open surgical repair. One such procedure is the Endo-Bentall procedure. The Endo-Bentall procedure aims to treat the same condition as the traditional Bentall procedure. However, it does so by eliminating the need to open a patient's chest cavity, Instead, an incision is made in a patient's groin and an Endo-Bentall stent device is tracked through the femoral artery and is positioned in the aortic root and valve. Although Endo-Bentall presents an incredible breakthrough in the treatment of aortic root and valve diseases, a number of issues still exist with the stent device used in the procedure and the method for delivering said stent graft.

The aortic root is a dynamic structure with documented variations in its dimensions during systole (pumping blood out of the heart chambers) and diastole (relaxation of the heart muscles, allowing the chambers to fill with blood). Thus, an Endo-Bentall stent graft must be able to accommodate this geometric fluidity by providing an adequate seal without inducing a fracture or tear and subsequent endoleak. With regards to dissections of the ascending aorta, there are expected acute increases in the mid-ascending aortic diameter up to 30%, which must be respected during graft selection. Oversizing the stent graft by more than 5% of the baseline media diameter can easily result in a new entry tear. The ascending aorta also has a short effective treatment length, compared to other vasculature within the body, and is restrained by the coronaries proximally and the brachiocephalic trunk distally. The pathology must be largely isolated to the root and/or proximal ascending aorta with a suitable landing zone of ‘healthy’ aorta proximal to the take-off of the first supra-aortic branch vessel. As a result, any sort of solution to issues stemming within the aortic root must address all of these issues and considerations.

Accordingly, there exists a need in the art for an Endo-Bentall stent graft with the ability to conform to the geometry and position of the aortic root, valve, and coronary arteries and a delivery device and method suited to implant such a stent graft.

### SUMMARY

According to certain aspects of the present disclosure, systems and methods are disclosed for a stent graft and deployment methods related to said stent graft. In one aspect of the disclosure, a medical device is disclosed. A medical device includes first expandable graft having a proximal and a distal end defining a graft lumen extending therebetween, the graft having a first portion defining a first diameter and the graft having a second portion defining a second, different diameter. The device includes a first tunnel lumen having a proximal end and a distal end defining a first tunnel lumen therebetween, the first tunnel lumen longitudinally aligned with the graft lumen, and a second tunnel lumen having a proximal end and a distal end defining a second tunnel lumen therebetween, the second tunnel lumen longitudinally aligned with the graft lumen. The proximal end of the first graft lumen is configured to couple with a valve, wherein the graft lumen trifurcates in a retrograde manner parallel with the first tunnel and second tunnel, the first tunnel configured to receive a first bridging stent and second tunnel configured to receive a second bridging stent.

In various embodiments, the first expandable graft includes a transition portion between the first portion and the second portion, wherein the transition section provides a gradual change in diameter from the first diameter to the second diameter.

In various embodiments, a first fenestration and a second fenestration are located on opposing sides of the transition portion, the first fenestration in fluid communication with the first tunnel lumen and the second fenestration in fluid communication with the second tunnel lumen.

In various embodiments, the angle between the first fenestration and second fenestration is approximately 100 degrees to approximately 140 degrees.

In various embodiments, the diameter at the distal ends of the first and second tunnel lumens is different than the diameter at the proximal ends thereof.

In various embodiments, the diameter of at least one of the first fenestration and second fenestration is adjustable.

In various embodiments, a first angled opening is proximal to the first fenestration and a second angled opening is proximal to the second fenestration.

In various embodiments, at least one of the first fenestration and second fenestration is surrounded by a ring.

In various embodiments, a first end of the ring is removably coupled to a control wire and a second end of the ring is coupled to the graft.

In various embodiments, a radial load is applied to the ring of at least one of the first fenestration and second fenestration, thereby increasing its diameter, when tension is applied via the control wire.

In various embodiments, the diameter of at least one of the first fenestration and the second fenestration is adjustable from approximately 5 mm to approximately 10 mm.

In various embodiments, at least one of the first fenestration and the second fenestration is surrounded by a set of radially extending struts.

In various embodiments, when a radial load is applied to the struts, the struts deform to expand the diameter of the at least one fenestration.

In various embodiments, when a radial load is removed from the struts, the struts constrict the diameter of the at least one of the first fenestration and second fenestration.

In various embodiments, at least one of the first fenestration and second fenestration is surrounded by an elastic fabric to adjust the diameter of said fenestration.

In various embodiments, the valve is coupled to the second portion of the first graft by at least one lock stent.

In various embodiments, the lock stent includes a tubular body composed of annular rings, each annular ring including a series of peaks and valleys, an elongated portion extending longitudinally from each peak.

In various embodiments, each elongated portion of the lock stent is curved radially inward.

In various embodiments, a second lock stent is positioned spaced apart from a first lock stent such that each peak of the first lock stent is longitudinally aligned with each peak of the second lock stent wherein the elongated portions of the first and second lock stent extend along opposite directions from one another.

In various embodiments, a second graft is at least partially positioned within the first graft such that a fenestration formed in the second graft is at least partially aligned with a fenestration formed in the first graft, and wherein the second fenestration is surrounded by a radiopaque ring.

In various embodiments, the first fenestration and second fenestration are concentrically aligned.

In various embodiments, the first fenestration is surrounded by a radiopaque ring.

In various embodiments, a longitudinal length of the second graft is less than a longitudinal length of the first graft.

In various embodiments, a diameter of the fenestration formed in the second graft is less than a diameter of the fenestration formed in the first graft.

In various embodiments, the proximal end of the first portion includes a foldable pleat forming a cavity between an inner surface of the graft and the folding pleat.

In various embodiments, the cavity is configured to receive a portion of the valve.

In various embodiments, the pleat includes at least one locking mechanism.

In various embodiments, the at least one locking mechanism includes a magnet disposed on the foldable pleat and a mating magnet disposed on an exterior surface of the first graft.

In various embodiments, the at least one locking mechanism includes a male button disposed on the first graft and a female button disposed between adjacent struts of an annular stent of the valve, the male button received within the female button in a locked configuration.

In another aspect of the disclosure, a method of assembling a medical device prior to implantation is provided. An expandable graft having a proximal and a distal end defining an internal graft lumen extending therebetween is provided. The graft has a first portion defining a first diameter, a second portion defining a second diameter. A valve is inserted within the internal graft lumen, the valve and expandable graft having an overlapping portion. A first arm of a fixation tool is at least partially inserted within the internal graft lumen, with a second arm of the fixation tool disposed on an exterior of the expandable graft, at least one of the first arm and second arm including an anchor disposed thereon. The anchor is inserted through the overlapping portion of the valve and expandable graft.

In various embodiments, the anchor pierces the expandable graft within the overlapping region of the valve and expandable graft.

In various embodiments, the anchor is disposed on the first arm inserted within the graft lumen.

In various embodiments, the first arm and second arm of the fixation tool are displaced towards each other to apply a force to the anchor.

In various embodiments, the anchor has first and second ends, the second end is deformed upon inserting the anchor through the overlapping portion of the valve and expandable graft.

In various embodiments, the anchor includes a male connector and a female connector, the male connector coupled to the second arm of the fixation tool and the female connector coupled to the first arm of the fixation tool.

In another aspect of the disclosure, a medical device is provided. The medical device includes an expandable graft having a proximal and a distal end defining an internal graft lumen extending therebetween, the graft having a first portion defining a first diameter and a second portion defining a second, different diameter. The medical device includes a first elongated constraint and a second elongated constraint disposed at opposing sides of the expandable graft, wherein the first and second elongated constraint include a first end and second end thereby defining a length extending therebetween. A valve is disposed at a proximal end of the expandable and at least partially inserted within the internal graft lumen to define an overlapping portion, wherein the first elongated constraint and second elongated constraint couple the valve to the expandable graft.

In various embodiments, one end of the first and second elongated constraint is fixed to the expandable graft and the second end is free.

In various embodiments, at least one of the first elongated constraint and second elongated constraint are fixed to an inner surface of the expandable graft.

In various embodiments, at least one of the first elongated constraint and second elongated constraint are fixed to an exterior surface of the expandable graft.

In various embodiments, the valve is secured to the expandable graft by weaving the free end of the first elongated constraint and the free end of the second elongated constraint through a lattice structure of the graft and valve within the overlapping portion.

In another aspect of the disclosure, a medical device is provided. The medical device includes an expandable graft having a proximal and a distal end defining a graft lumen extending therebetween, at least one fenestration located on a circumferential side of the expandable graft and in fluid communication with the graft lumen, and wherein the diameter of at least one of the first fenestration is adjustable.

In various embodiments, the at least one fenestration is surrounded by a ring.

In various embodiments, a first end of the ring is removably coupled to a control wire and a second end of the ring is coupled to the graft.

In various embodiments, a radial load is applied to the ring of the at least one fenestration, thereby increasing its diameter, when tension is applied via the control wire.

In various embodiments, the diameter of at the least one fenestration is adjustable from approximately 5 mm to approximately 10 mm.

In various embodiments, the at least one fenestration is surrounded by a set of radially extending struts.

In various embodiments, when a radial load is applied to the struts, the struts deform to expand the diameter of the at least one fenestration.

In various embodiments, when a radial load is removed from the struts, the struts constrict the diameter of the at least one fenestration.

In various embodiments, the at least one fenestration is surrounded by an elastic fabric to adjust the diameter of said fenestration.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is an illustration of a side view of an exemplary stent graft, in accordance with various embodiments of the present disclosure.

FIG. 2A-E are illustrations of proximal, distal, perspective, and a close-up perspective view of the exemplary stent graft, in accordance with various embodiments of the present disclosure.

FIG. 3A and FIG. 3C are front views of an exemplary stent graft, in accordance with various embodiments of the present disclosure.

FIG. 3B is bottom view of an exemplary stent graft in accordance with various embodiments of the present disclosure.

FIG. 4 is an illustration of an exemplary stent graft before insertion of a valve at the second portion and after insertion of a valve at the second portion, in accordance with various embodiments of the present disclosure.

FIG. 5 is an illustration of an exemplary curved wire stent, in accordance with various embodiments of the present disclosure.

FIG. 6 is an illustration of an exemplary fenestration ring attached to a stent graft via sutures, in accordance with various embodiments of the present disclosure with a fenestration ring on the main body of the graft presenting a clear visual marker of the exit of the fenestration that aids in cannulation (e.g. pre-cannulated fenestration).

FIG. 7 is an illustration of an exemplary stent graft provided with circumferential constraining sutures positioned at the level of one or more fenestrations, in accordance with various embodiments of the present disclosure.

FIG. 8 are illustrations of exemplary articulating fenestrated stent grafts, in accordance with the present disclosure.

FIG. 9 is an illustration of an exemplary fenestrated stent graft having a pair of overlapping fenestrations of differing orifice sizes, in accordance with the present disclosure.

FIG. 10 is an illustration of a perspective view of an exemplary coiled ring for adjusting the diameter of a fenestration, according to various embodiments of the present disclosure.

FIG. 11 is an exemplary series of steps involved in adjusting the diameter of a fenestration with an exemplary coiled ring and control wire, according to various embodiments of the present disclosure.

FIG. 12 is an illustration of a perspective view of an exemplary 3D mesh structure for adjusting the size of a fenestration, according to various embodiments of the present disclosure.

FIG. 13 is a front view of a wire strut fenestration adjustment mechanism, in accordance with various embodiments of the present disclosure.

FIG. 14 is a side view of an exemplary valve and stent graft attached by a series of locking stents, in accordance with various embodiments of the present disclosure.

FIG. 15 illustrates a front view of an exemplary lock stent, in accordance with various embodiments of the present disclosure.

FIGS. 16A and 16B are exemplary stent grafts before and after the addition of a locking stent to secure a valve thereto, in accordance with various embodiments of the present disclosure.

FIG. 17 is a transparent perspective view of an exemplary stent graft and valve attached by a button/magnet mechanism.

FIG. 18A-B depict a section view of the steps involved in securing a valve to a stent graft by a button/magnet style valve attachment mechanism taken along the y-axis.

FIG. 19 is a close-up view of the positioning of the attachment mechanism of FIGS. 18A and B, in accordance with various embodiments of the present disclosure.

FIG. 20 is a sectional view of an alternative embodiment of a stent graft and valve attachment mechanism taken along the z-axis.

FIGS. 21 and 22 are perspective views of the front and back of another embodiments of an attachment mechanism for a securing a valve to a stent graft, in accordance with various embodiments of the present disclosure.

FIG. 23 is a perspective view of a male and female anchor fixation tool, in accordance with various embodiments of the present disclosure

FIG. 24A-D is depicts illustrations of the series of steps involved in attaching a valve to a stent graft with an anchor and stamp assembly via a fixation tool, in accordance with various embodiments of the present disclosure.

FIG. 25 is a top sectional view of an installed anchor and stamp assembly, in accordance with various embodiments of the present disclosure.

FIG. 26A-C are illustrations of a side view of a fabric constraint valve attachment mechanism, in accordance with various embodiments of the present disclosure

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

When reference is made herein to an aortic prosthesis, such as a “stent graft,” “prosthesis,” “stent graft prosthesis,” “vascular prosthesis” or other prostheses to be delivered or implanted in a patient, the word “proximal” means that portion of the prosthesis or component of the prosthesis that is relatively close to the heart of the patient, while “distal” means that portion of the prosthesis or component of the prosthesis that is relatively far from the heart of the patient.

When, however, reference is made to a delivery system or a component of a delivery system employed to deliver, or implant, a prosthesis, the word, “proximal,” as that word is employed herein, means closer to the patient receiving the endoprosthesis. When reference is made to a delivery system where a component of the delivery system is “distal,” as that term is employed herein, means further away from the patient and closer to the physician/operator using the deployment system.

For clarity, the word “proximate” means “close to,” as opposed to the meanings ascribed to “proximal” or “distal” described above with respect to either the prosthesis or delivery system.

A stent graft is an implantable device made of a tube-shaped surgical graft covering and a balloon expandable or self-expanding frame, where the surgical covering is made from a textile (PET, ePTFE, Nylon etc) and the frame is made from metals such as stainless steel, cobalt chromium, Nitinol etc. In many designs, the stents are attached to textile using a surgical grade suture that can be made from multiple materials such as PET, UHMWPE, Nylon etc. Finally, stent-grafts usually contain additional metallic elements intended to allow visualization under fluoroscopy (continuous x-ray). The stent graft is placed inside a blood vessel to bridge a weakened section of aortic wall or a partially or fully ruptured location of aortic wall, for example, an aneurismal, dissected, transected or other diseased segment of the blood vessel, and, thereby, either exclude the hemodynamic pressures of blood flow from the weakened segment of the blood vessel or prevent blood exiting the true lumen of the aorta through a partial or complete rupture of the blood vessel.

In selected patients, a stent graft advantageously eliminates the need to perform open thoracic or abdominal surgical procedures to treat diseases of the aorta and eliminates the need for total aortic reconstruction. Thus, the patient has less trauma and experiences a decrease in hospitalization and recovery times. The time needed to insert a stent graft is substantially less than the typical anesthesia time required for open aortic bypass surgical repair, for example.

Surgical and/or endovascular grafts have widespread use throughout the world in vascular surgery. There are many different kinds of vascular graft configurations. Some have supporting framework over their entirety, some have only two stents as a supporting framework, and others simply have the tube-shaped graft material with no additional supporting framework.

Current stent grafts are designed with a memory shape at the point of manufacturing (Examples of such “memory” shape materials are disclosed in U.S. Pat. No. 6,306,141 which is hereby incorporated by reference in its entirety). As such, these stent grafts are constantly trying to reform to the memory shape. Once the stent is exposed from the sheath during deployment, the stent expands to a larger shape than the delivery sheath. With the stent graft at its expanded diameter, the physician will be able to add the fenestrations using the template tooling.

### Endo-Bentall Stent Graft

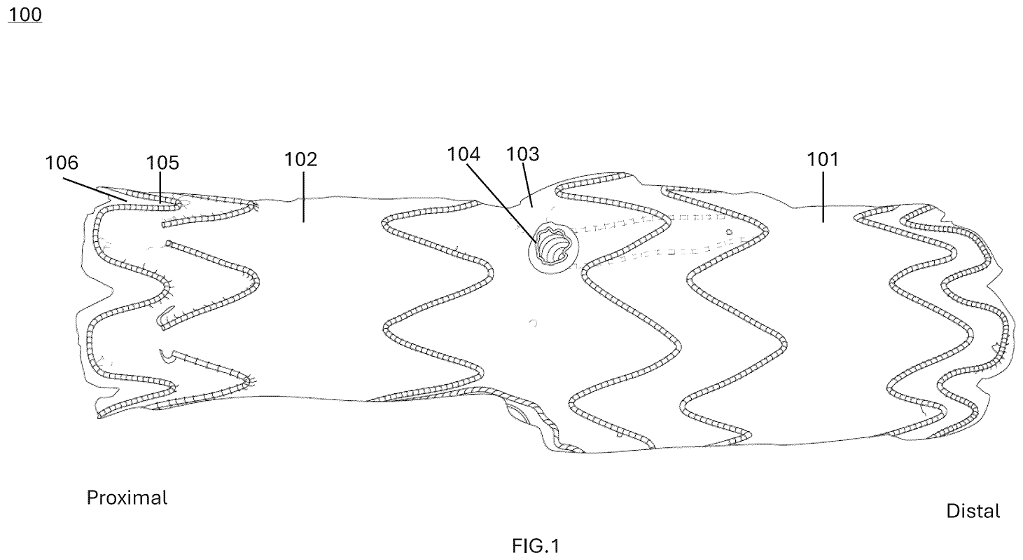

FIG. 1 is an illustration of a side view of an exemplary stent graft, in accordance with various embodiments of the present disclosure.

An exemplary stent graft 100 can include a tubular fabric member having at least a first and second ends, and a length extending between the first and second ends. A first portion 101 of the stent graft defines a first diameter and a second portion 102 defines a second diameter. The second portion 102 can be adapted to hold a valve, described in further detail below. The diameter of the first portion 101 can be larger than the diameter of the second portion 102 of the stent graft. A transition portion 103 may be positioned between the first portion 101 and second portion of the stent graft, where the diameter of the transition portion 103 gradually changes from the diameter of the first portion 101 to the diameter of the second portion 102; alternatively the transition portion can be configured with a step-change in diameter between first and second portions. In various embodiments, the graft 100 may include a plurality of wire stents 105 that conform to the shape of the inner surface of a patient's vasculature. For example, and without limitation, a plurality of wire stents 105 may be sutured onto a fabric 106 graft to create a rigid structure. Exemplary stent 105 shapes and configurations can conform to any of those known in the art.

In various embodiments, the diameter of the first portion 101 may correspond to the diameter of a patient's ascending aorta. In various embodiments, the diameter of the second portion 102 can correspond to the diameter of a valve meant to replace a patient's aortic valve.

In various embodiments, the length of the first portion 101 of the stent graft can correspond to the length of a patient's ascending aorta. In various embodiments the length of the first portion 101 can correspond to the length of the entire aortic arch (i.e., the length of the ascending and descending aorta).

In various embodiments, the length of the second portion 102 can correspond to the length of a patient's aortic valve, and is configured to receive a prosthetic valve coupled thereto. In various embodiments, the length of the second portion 102 can be shorter or longer than the length of a patient's aortic valve.

In various embodiments, the diameter of the first portion 101 and second portion 102 may remain consistent along their lengths. In various embodiments, the diameter of the first portion 101 and second portion 102 may vary along their lengths to accommodate any inconsistencies or variations within a patient's vasculature.

A pair of fenestrations 104, which may be adapted to receive a pair of bridging stents, are disposed on opposing sides of the transition portion 103, corresponding to the positions of a patient's coronary arteries, and extend through the fabric of the graft. In various embodiments, the fenestrations 104 may be disposed on the proximal end of the transition portion 103. In various embodiments, the fenestrations 104 may be disposed on the distal end of the distal end of the transition portion 103. The fenestrations can be oriented to be radially-outward facing (e.g. perpendicular or normal to the longitudinal axis of the graft), and/or oriented at an angle (e.g. oblique to the longitudinal axis of the graft). In various embodiments, the fenestrations 104 may be disposed on the first portion 101. In various embodiments, the fenestrations 104 may be disposed on the second portion of the stent graft. In operation, a user (i.e., a clinician or surgeon) can determine the proper positions for the fenestrations 104 based on a patient's vasculature and more particularly, based on the positions of the coronary arteries, and make the fenestrations accordingly.

In various embodiments, the fenestrations 104 can be fixed in size and shape without option for change. In various embodiments, the fenestrations 104 can be adjustable in size and shape.

Once fenestrations 104 are made, the stent graft 100 can be reloaded into the deployment device and ready for insertion within a user's ascending aorta and aortic valve in accordance with various known methods in the art. For example, and without limitation, the stent graft may be compressed within a sheath and tracked through a patient's vasculature until it reaches the correct position. Then, the sheath may be pulled back or retracted to expose and place the stent graft. In various embodiments, once the stent graft 100 is placed at the desired position within a patient's anatomy, a valve may be secured to the second portion 102 using one of a variety of suitable attachment methods, described below. In various embodiments, the valve may be secured to the stent graft 100 prior to delivery within the patient's vasculature.

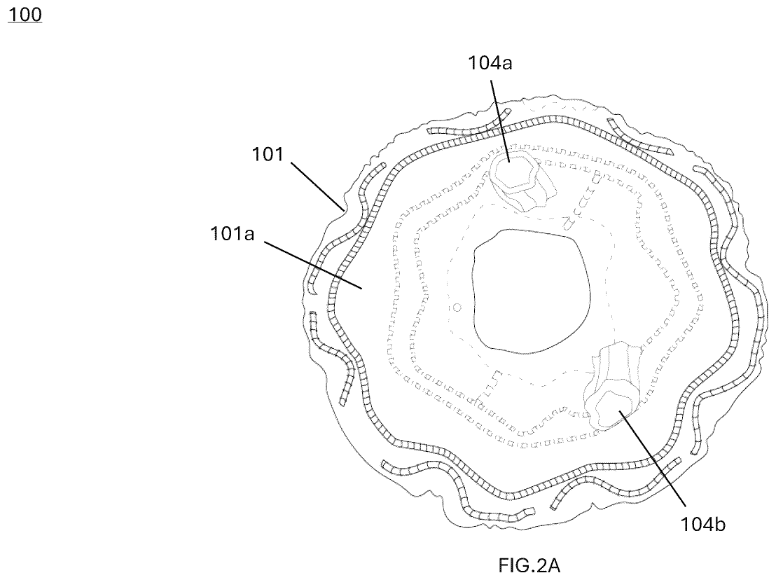

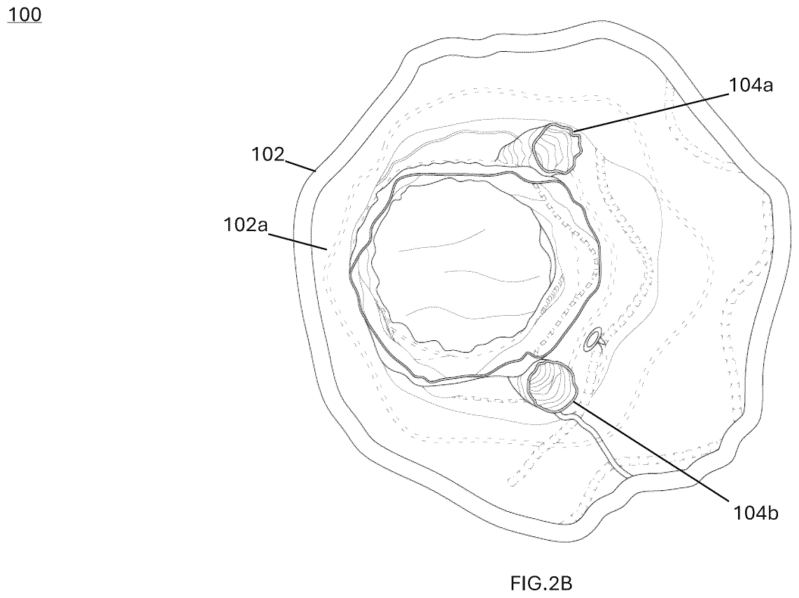

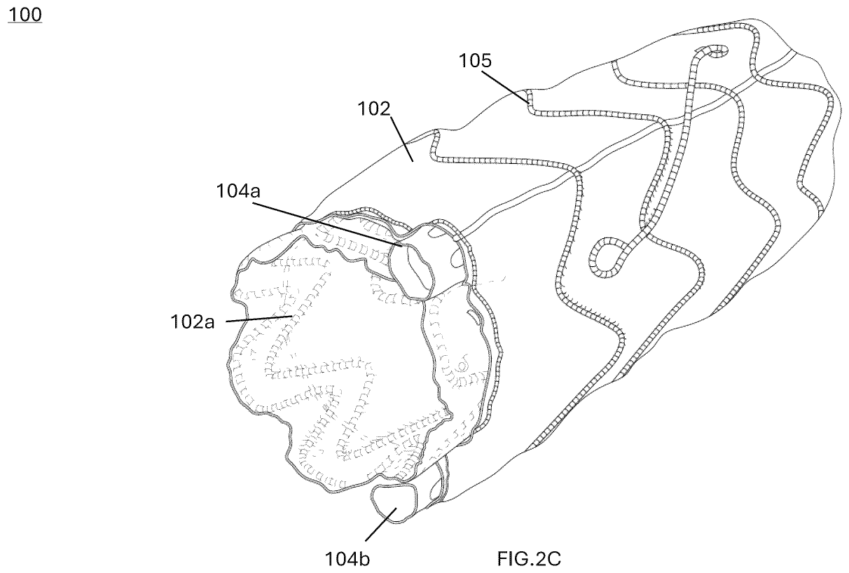

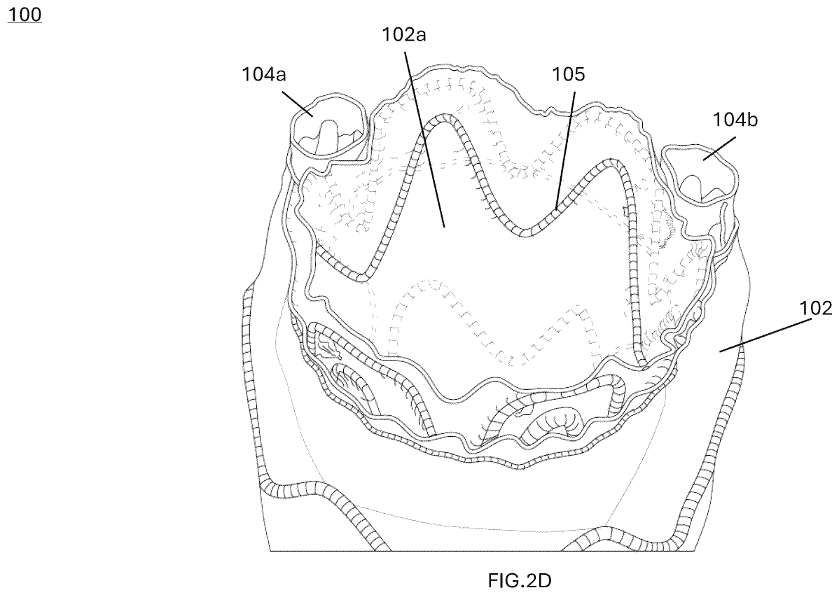

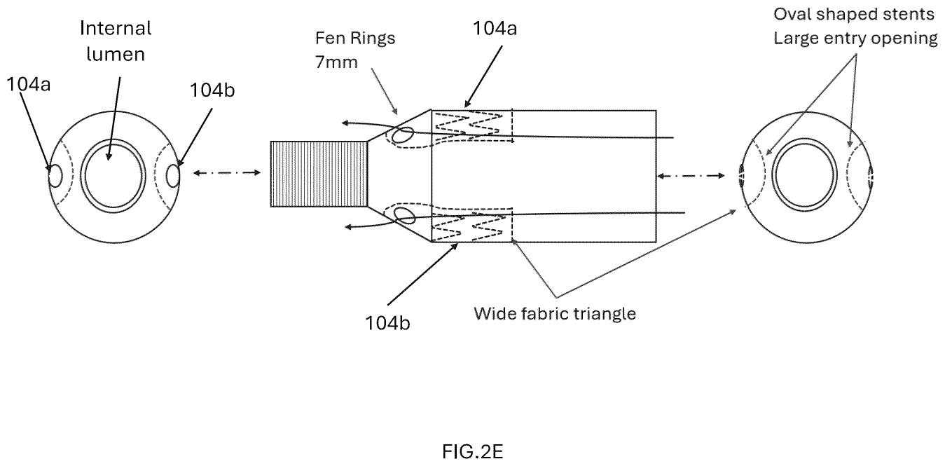

FIG. 2A-E are illustrations of top, bottom, perspective, and a close-up perspective view of the first portion of the exemplary stent graft, in accordance with various embodiments of the present disclosure. FIG. 2A-2E show that a first tube 104a and second tube 104b extend out from the pair of fenestrations 104 and along the length of the first portion 101 of the stent graft.

Reference to “tunnel” and “tube” may be used interchangeably throughout, and each tunnel/tube 104 has a respective tunnel/tube lumen extending therein; and the graft 101,102 also has a main/primary lumen extending along the length of the graft member (with the tunnel/tube 104 lumens radially offset from the central longitudinal axis of the graft, e.g. each tunnel can be diametrically opposed to each other and coupled to the graft 101, 102—an exemplary embodiment illustrated in FIG. 2E. In some embodiments, as shown in FIG. 2A-D, the orifice of tubes 104 are located at the terminus of the graft. In various embodiments, the first tube 104a and second tube 104b extend generally parallel in the direction of the first portion 101 of the stent graft, such that the distal portion of the first portion 101 of the graft lumen trifurcates in a retrograde manner parallel with the tubes 104. In other words, at the proximal end of the device the main lumen of the graft is separated from the first and second tubes, and at the distal end of the device all three lumens are fluidly coupled. The diameter of the first tube 104a and second tube 104b may be large enough to support guide wires (FIG. 2E) that extend into the coronary arteries (after which additional grafts or anchoring stents can be deployed or cannulated with respect to the tubes 104a,b). In various embodiments, the first tube 104a and second tube 104b may extend across the entire length of the first portion 101. In various embodiments, the first tube 104a and second tube 104b may only extend across a portion of the length of the first portion 101.

In various embodiments, the diameter at the distal portion of the first tube 104a and second tube 104b is greater than the diameter at the proximal portion of the first tube 104a and second tube 104b (FIG. 2E). This aids in cannulation of the tunnels and into the coronary as the guidewire exits the proximal end of the tunnel 104. Fenestration rings can be incorporated at the exit of the tunnel to provide a clear indication of wire location as it exits the main body of the graft.

In various embodiments, the diameter at the distal portion of the first tube 104a and second tube 104b is the same as the diameter at the proximal portion of the first tube 104a and second tube 104b. In various embodiments, the diameter at the distal portion of the first tube 104a and second tube 104b is smaller than the diameter at the proximal portion of the first tube 104a and second tube 104b.

In various embodiments, the first tube 104a and second tube 104b may be fixed to the interior of the walls of the first portion 101 of the stent graft 100. In various embodiments, the first tube 104a and second tube 104b may be removably attached to the interior of the walls of the first portion 101 of the stent graft 100. In various embodiments, the first tube 104a and second tube 104b may be attached to the interior of the walls of the first portion 101 of the stent graft 100 by adhesive bonding, suturing, stapling and/or any other known methods to one of ordinary skill in the art. In various embodiments, the stent graft 100 may be packaged such that the first tube 104a and the second tube 104b are separate from the stent graft 100, requiring a user attach the components together prior to implanting the graft within a patient. In various embodiments, the stent graft 1000 may be packaged such that the first tube 1004a and the second tube 1004b are pre-fixed to the stent graft, allowing a user to implant the graft within a patient without any modification, except for creating fenestrations that open into the first and second tube (104a, 104b).

In various embodiments, the diameter of the first tube 104a and second tube 104b can vary along their lengths. In various embodiments, the diameter of the first tube 104a and second tube 104b is consistent along the entire length.

In various embodiments, the diameter of the first tube 104a and second tube 104b can match the diameter of the fenestrations 104 of which they correspond to. In various embodiments, the diameter of the first tube 104a and second tube 104b can differ from the diameter of the fenestrations 104 of which they correspond to.

In various embodiments, the cross section of the first tube 104a and second tube 104b can be circular. In various embodiments, the cross section of the first tube 104a and second tube 104b can be non-circular in shape to conform to all of the surfaces of a patient's vasculature.

### Covered Windows with Retrograde Tunnels

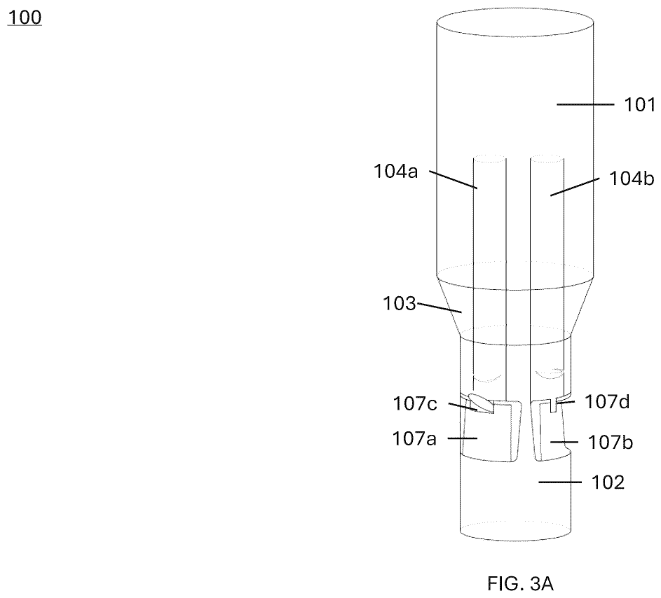

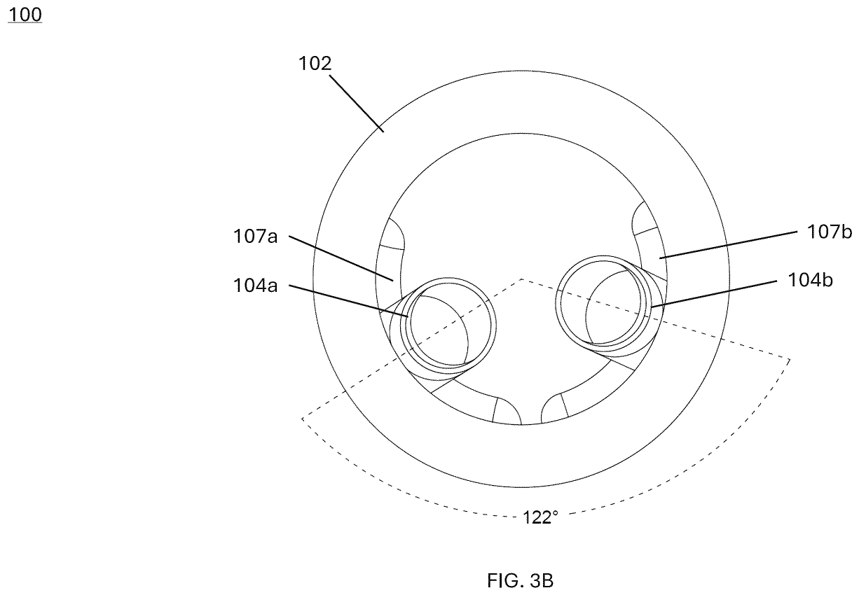

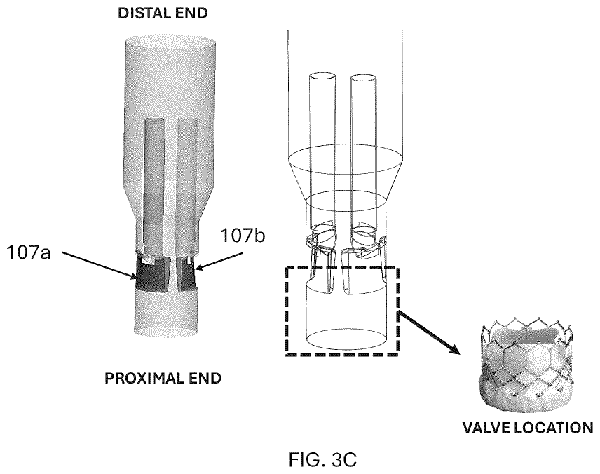

FIG. 3A is a transparent side view of an exemplary stent graft 100, in accordance with various embodiments of the present disclosure. In various embodiments, the stent graft 100 can include a first angled opening 107a and a second angled opening 107b proximal to the first and second fenestration 104, respectively. These angled openings can serve as ramp-like structure to facilitate alignment and insertion of the bridging stents within the first and second tubes.

In accordance with an aspect of the disclosure, a stent graft with internal tunnels connected to fenestration windows that are covered at the end opposite the tunnels provide a barrier between the bridging stents and the valve, effectively preventing leaks and ensuring secure, reliable deployment. Also, the fenestration windows facilitate easier alignment and landing of the stent graft relative to target branch vessels, thus reducing the difficulty of landing in the correct location.

Additionally, a valve is designed to be inserted into the proximal end of the graft (FIG. 3C). Once in position, the valve can be reloaded into a delivery system for deployment. This design ensures effective sealing and optimal functionality while maintaining ease of use during the stent graft's insertion and placement.

The first angled opening 107a and second angled opening 107b can be formed with a ramp-like geometry, having a slanted face with tapered thickness along the longitudinal axis, such that the flaps angle backwards in order to accommodate the opening of the first tube 104a and second tube 104b. The first angled opening 107a and second angled opening 107b can include a cut out (107c, 107d) at the portion aligned with the opening of the first tube and second tube at the outer surface of the stent graft. In various embodiments, the cut outs (107c, 107d) may be square shaped. In various embodiments, the cut outs (107c, 107d) may be shaped as a semi-circles corresponding to the shape of the first tube 104a and second tube 104b so as to serve as coverings for the underlying windows or openings to the first and second tubes 104a, 104b.

In various embodiments, the first and second angled opening (with window covering flap) 107a, 107b can be disposed on the outer surface of the stent graft 100 by forming a cut out around the opening of the first and second tube (104a, 104b) and adhering perimeters of the angled openings to the cut-outs. In various embodiments, the first angled opening 107a and second angled opening 107b can be attached to the stent graft 100 via any suitable method known to one of ordinary skill in the art. In various embodiments, the stent graft 100 can be packaged such that first angled opening 107a and second angled opening 107b are pre-fixed to the stent graft allowing the user to implant the graft without modification. In various embodiments, the stent graft 100 can be packaged separately from the first angled opening 107a and second angled opening 107b such that the user can attach the flaps to the graft prior to implantation of the graft. Packaging the components separately may allow a user to make modifications to the graft prior to implantation, such that the graft fits to a patient's vasculature more precisely.

In operation, an implanted stent graft 100 and attached valve (FIG. 3C) can support blood flow from the heart as it pumps blood from aortic valve into the ascending aorta. Guidewires extending from the first portion of the stent graft 101 to the second portion 102, exit through the first and second fenestration 104. The first angled opening 107a and second angled opening 107b surrounding the first and second fenestration 104 support the guidewires as they exit from the first and second fenestration 104 and into the coronary arteries, where blood is supplied to the musculature of the heart.

FIG. 3B is bottom view of an exemplary stent graft in accordance with various embodiments of the present disclosure. As can be seen in FIG. 3B, the first tube 104a and second tube 104b can be spaced apart from one another by about 122 degrees. As discussed, the first tube 104a and second tube 104b lead to the coronary arteries of a patient. Thus, their positions correspond to those of the coronary arteries. In various embodiments, the angle between the first tube 104a and second tube 104b may be more than 122 degrees, depending on a specific patient's anatomy. The angle between the first tube 104a and second tube 104b may be less than 122 degrees, depending on a specific patient's anatomy.

Although FIG. 3B illustrates an angle of about 122 degrees between the first tube 104a and second tube 104b, in various embodiments, the angle between the centerlines of the first tube 104a and second tube 104b can be from 100 degrees to 140 degrees, inclusive. Any value or sub-range within 100-140 degrees may be selected to match a patient-specific coronary geometry.

In various embodiments, the first tube 104a and second tube 104b can extend longitudinally through the first portion of the stent graft 101. In various embodiments, the first tube 104a and second tube 104b can extend at an angle through the first portion of the stent graft 101.

### Valve Attachment

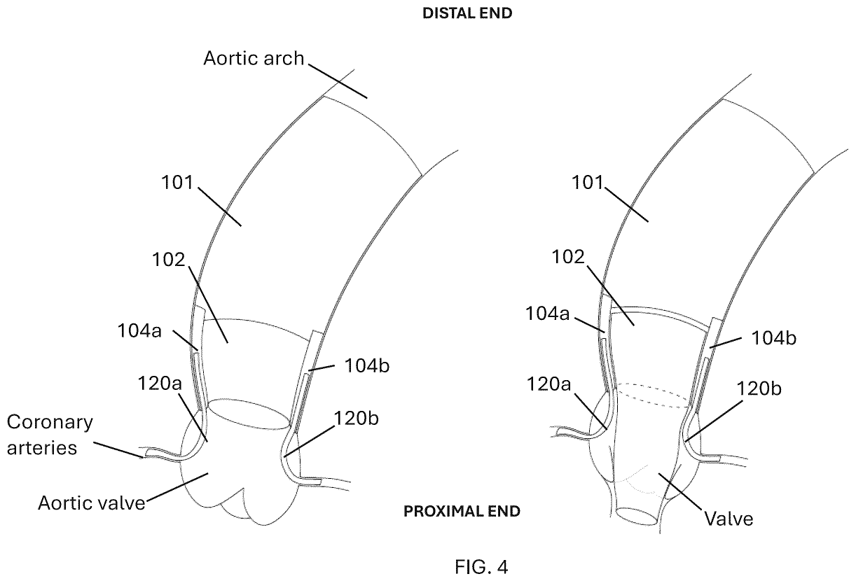

FIG. 4 is an illustration of an exemplary stent graft before insertion of a valve at the second portion 102 and after insertion of a valve at the second portion 102, in accordance with various embodiments of the present disclosure. FIG. 4 shows that a valve (which can itself include an elongated stent graft portion) is coupled with the second portion 102 of the stent graft 100. Guidewires (120a, 120b) extend out of the first and second tube (104a, 104b) of the graft member and into the first and second coronary artery. In some embodiments, the tunnels/tubes 104a, b can have distal ends that terminate at a location between proximal and distal ends of the main graft (e.g. the distal ends of tunnels/tubes 104a, b can terminate at a location distal to the distal end of the valve. For purpose of illustration and not limitation, the distal ends of the tunnels/tubes 104a, b can terminate approximately 5˜20 mm from the distal end of the valve. In various embodiments, as described below, the valve 1010 may be fixed to the stent graft 100 by a variety of methods (e.g. sutures, mechanical interlock/deformation, interference fit, adhesives, etc.).

In various embodiments, the second portion of the stent graft 102 can extend partially into the aortic valve (such that a portion of the valve circumferentially overlaps and circumscribes the graft 102—as shown by the dashed line on the right side of FIG. 4). In some embodiments, the stent graft 102 receives the aortic valve (such that a portion of the graft 102 circumferentially overlaps and circumscribes the valve member).

The amount of overlap can vary depending on the amount of coupling desired, provided the proximal end of the stent graft 101,102 does not extend to interfere with the flow of blood through valve leaflets. In some instances, the amount of overlap can be approximately 2 mm, or more. The larger the amount of overlap, the stronger the coupling between the two components.

In various embodiments, the second portion of the stent graft 102 can terminate before maximum range of motion of the leaflets of aortic valve (thus avoiding obstruction of the valve function).

The stent graft 101, 102 can have one or multiples tunnels 104 positioned at different levels (e.g. radial distance from the internal diameter of the graft) along the length of the stent graft. The internal lumen can include reinforcing stents (e.g. for enhanced rigidity and more certain maximum expansion diameter); or alternatively be free of any reinforcing stent structures (e.g. to minimize undesired interaction with the valve member). Similarly, the external graft 101,102 can include reinforcing stents.

In some embodiments, the internal lumen of stent graft 101, 102 is designed to be smaller than the external graft lumen (e.g. by approximately 3 mm, or more). One or multiples internal tunnels 104 can be positioned between the internal lumen and external graft. The benefit is to create a stent graft that has internal tunnel(s) and the graft can be extended using the internal lumen as a modular docking section. The internal lumen helps reduce the potential for endoleak and protects the tunnel(s) 104 from being crushed. Again, the amount of overlap between the two components (graft and valve) controls the amount of extension (e.g. longitudinal displacement of the graft 101,102 relative to the valve) that is available to the physician during deployment to best match the patient's specific anatomy.

In accordance with an aspect of the disclosure, the operator (e.g. physician) can deploy the stent graft 100 to the desired location within the patient (e.g. approximately 10 mm distal to the coronary arteries). Next, one or more bridging stents can be deployed (over guidewires 120a,b) with the distal end of the bridging stents received within the tubes 104a,b. In some embodiments, half the length of the bridging stents can be received within the tubes 104a,b, with the remainder projecting outward from the proximal end of the graft 102. In some embodiments less or more length of the bridging stents can be received within the tubes 104a,b, the greater the length of bridging stent positioned within the tubes, 104a,b, the more rigidly fixed the stent graft 100 remains at the deployed location.

Next, the valve (which, as noted above, can be embodied in a second elongated graft distinct from graft 100) is deployed by inserting the distal end of the valve graft within the proximal end of the stent graft 102. The valve member and stent graft 100 can be coupled with the stent graft having a male connection and valve member having a female connection receiving the stent graft; or vice versa. In the exemplary embodiment shown in FIG. 4, the proximal end of the stent graft 100 has a male coupling (e.g. received within the inner lumen) with the valve member (as indicated by the broken line depicting the proximal end of the graft 100).

Accordingly, the valve can be fit into the second portion of the stent graft 102. In some embodiments the second portion 102 of the stent graft can extend longitudinally along the entire length of the aortic valve (and proximally beyond the valve). In various embodiments, the valve can be fit into the second portion of the stent graft 102 such that the second portion 102 only partially extends over the valve (e.g. the proximal end of the graft 102 is located distal to the proximal end of the aortic valve).

### Graft Geometry

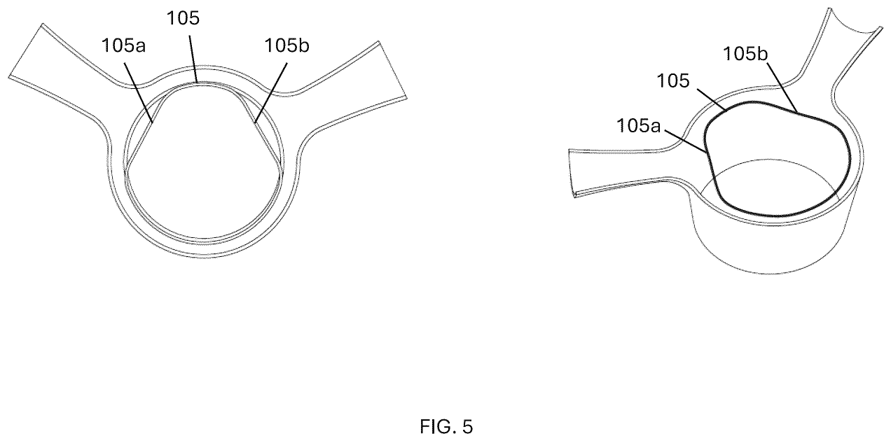

In various embodiments, the graft 100 may be composed of a series of radially expanding annular ring wire stents which have a non-circumferential shape (when expanded). As depicted in FIG. 5, at least one wire stent 105 embedded within graft 100 may be oriented inwards at locations (105a, 105b) such that the stent has a curved portion (at the apex location 105 as shown in FIG. 5) and planar section(s) 105a,b. These non-linear sides 105a,b, can be positioned on the graft 100 at locations corresponding to the patient's branching vessels. In various embodiments, these locations can correspond to the locations of the coronary ostium.

Curved, non-circular grafts can be advantageous in contexts where the site at which the stent graft is to be inserted possess variations in dimension and size. For example, and without limitation, portions of diseased vasculature can be atrophied or bloated, forming a non-circular, non-uniform cross section. These situations can call for stents which accommodate these anatomical variations in order to produce a tight seal between the graft and the diseased vasculature.

Further, the non-circumferential shape of the wires stents can allow for improved clearance of branch vessels while still providing adequate radial force and apposition to the vessel wall. The non-circular (e.g. planar) side 105a,b allows for any fenestration located thereon to be oriented with fenestration-orifice parallel to the opening of the branch vessel—thus a guidewire and/or bridging stent can be easily deployed in a straightforward manner and received within the branch vessel.

### Fenestration Ring and Sutures

In some embodiments, a fenestration can be created in a stent graft using any suitable method. For example, the graft material within the boundary of the fenestration edge/circumference can be cauterized. Cauterization can be advantageous in that it prevents unravelling of the stent graft fabric at the edge(s) of the fenestration. Additionally, or alternatively, a number of cuts can be created to segment the graft into the desired number, and shape, of fenestrations. In some embodiments the material cut from the graft is removed/discarded. In some embodiments, the material cut from the graft can remain partially attached, e.g. a flap, and folded back on the exterior surface of the graft.

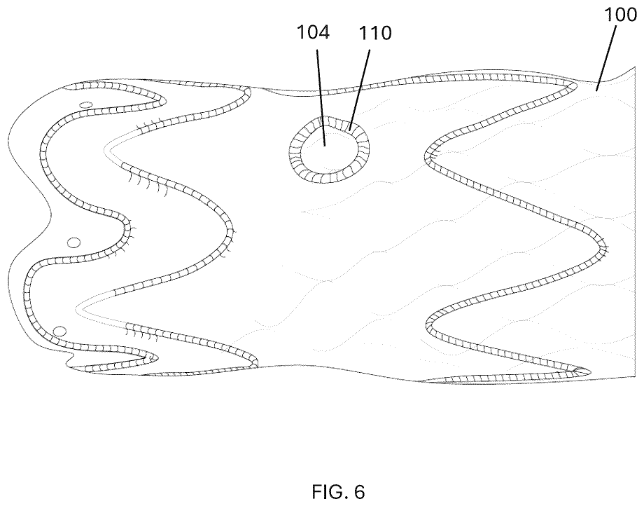

Additionally, a ring can be attached to the graft which circumscribes, or otherwise surrounds, the fenestration 104. An exemplary embodiment of a ring 110 surrounding the fenestration is shown in FIG. 6. In some embodiments the ring is attached to the graft after the fenestrations are formed. In other embodiments the ring is attached before the fenestrations are formed (but after the location of the fenestrations are identified on the graft). The fenestration ring can be formed of a radiopaque material to provide visibility of the location, and relative dimensions, of the fenestration. The ring can be provided as a closed loop, or alternatively an elongate string-like element that can be formed/shaped into a loop when attaching to the graft. In some embodiments the rings are elastic and/or deformable to adjust the size and shape to mimic the contours of the graft. Alternatively, the rings can be rigid to resist local deformations and retain a generally uniform shape. The fenestration rings can be provided in a variety of sizes, e.g. diameters (or widths if not circular) ranging from 4 mm˜12 mm; and/or of set sizes, such as 6 mm and 8 mm.

One or more suture threads can be used to attach the fenestration ring to the graft. The suture thread can also include a material with radiopaque properties, for visualization of the suture thread using radiographic imaging. The sutures can be made of synthetic filaments (single, poly, braided, etc.) and/or metallic thread. Alternatively, or in addition to the suture thread, fasteners such as, for example, staples, rivets, micro-rivets, adhesives, and/or welding can be used to secure the fenestration ring to the graft. Likewise, the fasteners can include or be formed of material with radiopaque properties such that the fasteners are visible using radiographic imaging.

In operation, the radiopaque fenestration ring 110 provides a distinct visual cue under imaging, enabling the operator to accurately align the fenestration with the target coronary artery ostium. The ring's structure ensures that the fenestration remains accessible, thereby facilitating cannulation and subsequent intervention.

### Circumferential Constraining Suture

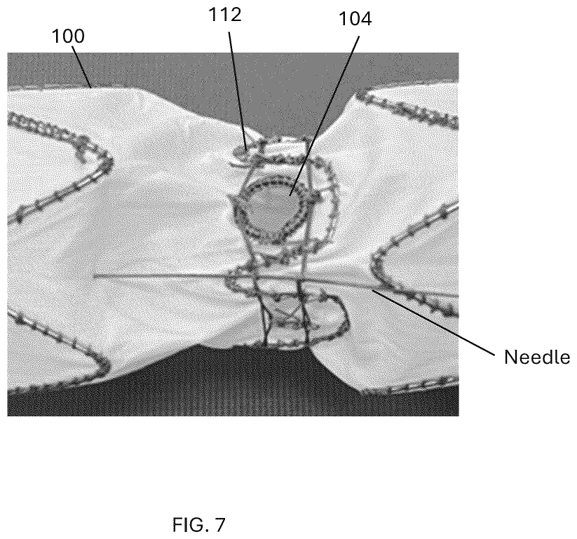

In various embodiments, a stent graft device 100 is provided with a circumferential constraining suture 112 positioned at the level of one or more fenestrations 104 as shown in FIG. 7. The circumferential constraining suture 112 is configured to temporarily reduce the diameter of the stent graft 100 at the fenestration level, thereby facilitating staged deployment of the device. This feature enables improved alignment of the fenestrations 104 with target anatomical structures, such as the coronary arteries, and assists in the cannulation process during endovascular procedures,

In various embodiments, the circumferential constraining suture 112 comprises a filament or thread that is circumferentially disposed around the external or internal surface of the stent graft 100 at the level of the fenestrations 104 and is applied by a needle or any other suitable device/method. The suture 112 may be secured to the graft material or to the stent framework at multiple attachment points, or it may be continuously looped around the circumference. The suture may be configured to maintain the stent graft in a partially constrained, reduced-diameter state prior to or during initial deployment. In some embodiments a pair of sutures 112 are applied, one on each side (proximal, distal) of the fenestration to apply equivalent radially constricting force to the graft thereby maintaining the orifice of the fenestration coplanar or conforming to the shape of the exterior surface of the graft.

Upon deployment within the vasculature, the circumferential constraining suture 112 maintains the stent graft 100 in a partially collapsed configuration at the fenestration level 104, allowing for precise positioning and rotational alignment of the fenestrations 104 relative to the target vessels. This constrained state facilitates the cannulation of the coronary arteries or other branch vessels through the fenestrations 104. In various embodiments, the suture 112 may be designed to be releasable, such that it can be removed or severed after successful cannulation and alignment, thereby allowing the stent graft to fully expand to its intended diameter and achieve apposition with the vessel wall.

In various embodiments, the circumferential constraining suture 112 may be constructed from materials that are absorbable or dissolvable within the body, such that the suture degrades over a predetermined period, automatically releasing the constraint and permitting full expansion of the stent graft. In other embodiments, the suture 112 may be configured with a pull-tab, loop, or other release mechanism that can be actuated by the operator via a catheter or guidewire. The suture 112 may also be integrated with locking or ratcheting mechanisms to allow for incremental or controlled release of the constraint.

In various embodiments, multiple circumferential constraining sutures 112 may be provided at different axial positions along the stent graft to facilitate staged or sequential deployment. The suture 112 may be positioned on the interior or exterior of the graft material, or may be embedded within the graft wall. The suture 112 may be continuous or segmented, and may be attached to the wire stents, the graft material, or both.

### Articulated Fenestrations

As discussed above, in some embodiments, the stent graft provides fenestrations (holes) to allow blood flow through the stent graft into side branch vessels. In some embodiments, the locations of the fenestrations are selected to avoid overlapping with the stent struts.

Generally, a precise fit between the fenestrations of the stent graft and the openings of the branch vessels is important both for ensuring the flow between the aorta and the branch vessels and for excluding the flow to the aneurysm. To ensure a precise fit, a stent graft for a particular patient is preferably fenestrated accordingly to that patient's particular anatomy.

As discussed above, a stent graft may include one or more fenestrations configured to accommodate one or more branch vessels when the stent graft is deployed in an aorta. A fenestrated portion of the stent graft includes at least one fenestration. The fenestrated portion may be located near a proximal end, a distal end or any portion of the stent graft where branch vessels need to be accommodated. The fenestration(s) can be circular, “scalloped” or U-shaped, and/or any other desired geometry to accommodate the patient's anatomy.

In various embodiments, the fenestrations in a stent graft may be of any suitable sizes or shapes. In typical embodiments, the fenestrations are sized and/or shaped to accommodate the corresponding branch vessel openings. In various embodiments, the peripherals or edges of the fenestrations may be reinforced wholly or partially to provide stability, for example, for anchoring of stent grafts into the branch vessels. In an embodiment, the peripheral of a fenestration may be stitched or sutured using wires. In another embodiment, the peripheral of a fenestration may be coupled (e.g., via stitches) to a ring or a similar support frame.

In various embodiments, the boundary of the fenestrations may be marked to facilitate visual tracking of the fenestrations during (final, or after the initial deployment of the graft wherein the fenestrations are formed in the graft) deployment of the stent graft. For example, the peripheral of the fenestrations may be sutured using gold wires or wires of other radio-opaque materials. Similarly, the location of the fenestration may be marked by one or more radio-opaque markers. Alternatively, radiopaque fenestration rings can be coupled to the graft to surround the fenestration and provide visibility to the physician. Such radio-opaque wires or markers may facilitate fluoroscopic visualization of the fenestrations during the repair procedure and allow a physician to locate the fenestration with respect to the corresponding branch vessel.

In accordance with another aspect of the disclosure, the fenestrations can be imparted onto the graft after the graft is partially deployed/expanded, using a fenestration template with fenestration location, dimensions, etc. based on scans of the specific patient's anatomy. An example of such a template and fenestration technique is disclosed in commonly owned and co-pending Patent Cooperation Treaty Application Number PCT/US24/44821, the entire contents of which are hereby incorporated by reference.

In accordance with another aspect of the disclosure, the fenestrations 104 can be formed as articulating fenestrations, an exemplary embodiment of which is shown in FIG. 8. Additional examples of movable fenestrations are disclosed in WO2005/034809 and US2010/0268327, the entire contents of each are hereby incorporated by reference.

In accordance with another aspect of the disclosure, the fenestrations can include flexibility/mobility such that the fenestration can articulate to position the radially outward orifice of the fenestration at any desired orientation. In the exemplary embodiment shown in FIG. 8, the fenestration is attached to a strip of fabric 104′ that extends radially outward from the graft outer diameter. This articulating portion 104′ spans the distance between the radially-inner fenestration ring and the radially-outer fenestration ring, thereby connecting the two rings. The articulating portion 104′ has a surface area that is greater than the plane of the tubular body between the edges and therefore will have excess material (as shown on right side of FIG. 8). This excess material allows the inner edge and the fenestration to move within the circumference of the outer edge—such that the inner fenestration ring need not be concentric or aligned with the outer fenestration ring. Similarly, the outer fenestration ring can be pitched or oriented at a different circumferential angle as compared to the inner fenestration ring. This allows for greater flexibility in cannulation and more precise positioning of the graft for a wider variety of anatomies. In some embodiments, the articulating portion 104′ can be formed as a semi-rigid arcuate (e.g. dome) shape.

The articulating portion 104′ can be formed from a separate piece of graft material that is stitched or otherwise attached to the graft body 100. The slack from the excess surface area of articulating portion 104′ allows the radially-outer fenestration ring to move within boundaries of the radially-inner fenestration ring. The excess material of the articulating portion 104′ can be evenly distributed in the annular area between the radially-inner fenestration ring and the radially-outer fenestration ring. This allows movement equally in all directions.

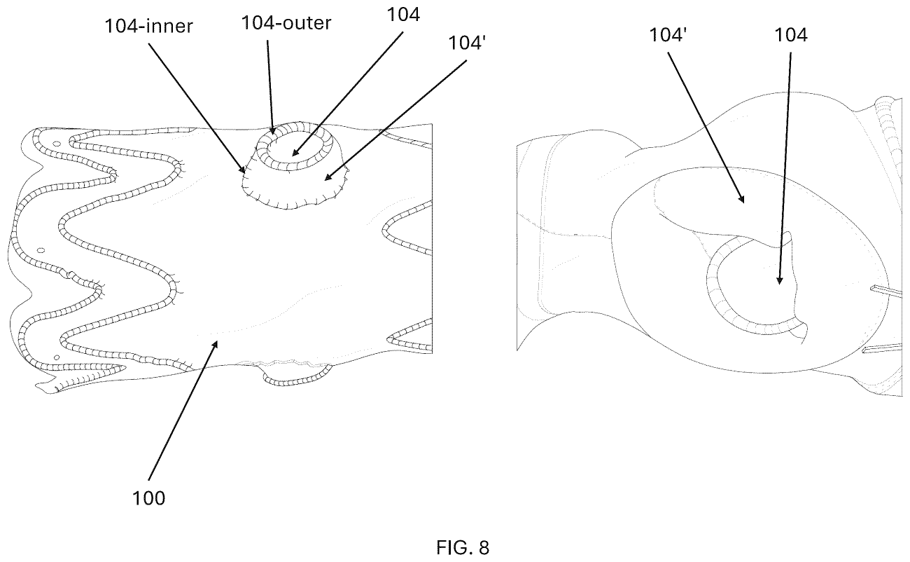

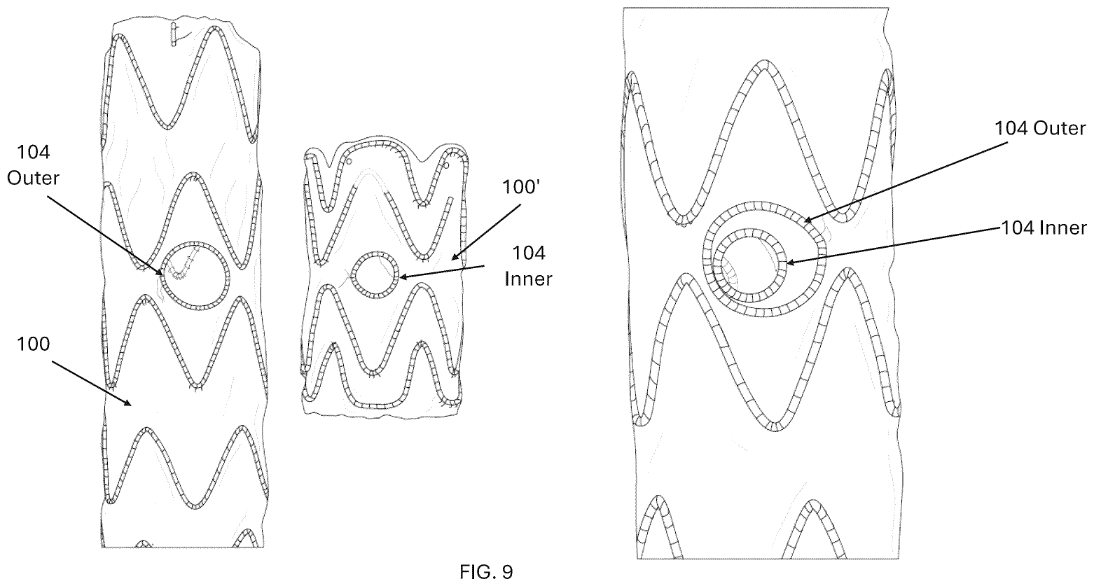

In accordance with another aspect of the disclosure, each (or select) fenestration locations can include a pair of overlapping fenestrations with varying orifice diameters. In the exemplary embodiment is shown in FIG. 9, a relatively large fenestration can be formed on the periphery of the graft, with another relatively small fenestration formed radially inward, as this transition from large to small fenestration size provides ease of alignment of the large fenestration with, e.g. coronary ostium, while the smaller diameter fenestration provides more secure coupling/positioning of the bridging stents deployed therein. For purpose of illustration and not limitation, in an exemplary embodiment the outer/larger fenestrations can have a ration of about 2:1 in diameter size, e.g. the outer fenestration can have a diameter of approximately 5 mm˜15 mm, while the inner fenestration can have a diameter of approximately 2 mm˜7 mm.

This design provides a main graft 100 with at least one stent and a large opening 104 on the lateral side of the graft. The large opening can be made smaller by using a second graft 100′ with a smaller opening (as shown on left side of FIG. 9). The second graft 100′ is designed to be longitudinally shorter than the first graft 100 and therefore easier to rotate and move caudally and cranially. Also, the second graft 100′ is designed to create a seal with the first graft 100 to prevent leakage around the large opening of the first graft 100. In some embodiments, the second graft 100′ can use one or multiple stents and have a larger internal lumen diameter than the first graft 100. The second graft 100′ opening 104 is designed to be positioned within the first graft 100 large opening 104, with the second graft 100′ is used to reduce the size of the opening 104 to the final desired opening size.

In addition to the added flexibility/mobility the fenestration, positioning the inner fenestration ring 104 off the main body of the graft first graft 100 presents a clear visual marker of the exit of the fenestration that aids in cannulation. Further, the second/inner graft 100′ can be repositioned (e.g. rotated, translated longitudinally) with respect to the first/outer graft 100 to adjust the relative positioning between the two fenestrations 104, as desired.

### Control Wire Adjustable Fenestration Size

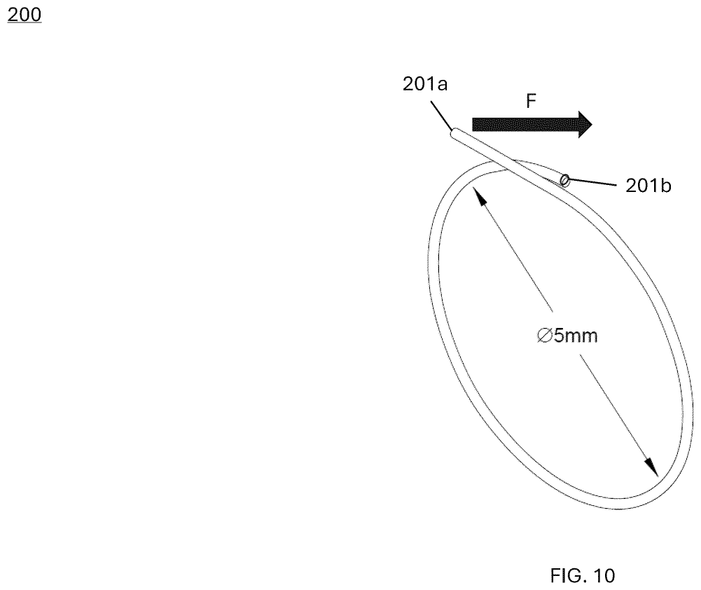

In various embodiments of the present disclosure, it may be advantageous to be able to adjust the size of fenestrations on the stent graft, which can be particularly advantageous for accommodating bridging stents while maintaining alignment with the patient's anatomy (e.g. coronary ostium). FIG. 10 is an illustration of a perspective view of an exemplary coiled ring and control wire for adjusting the diameter (or width if not circular; artisans will understand that any reference to “diameter” herein can equally apply to a “width” of a fenestration in non-circular embodiments) of a fenestration, according to various embodiments of the present disclosure. In various embodiments, a fenestration of a stent graft may be lined with a coiled ring 200. For example, and without limitation, a coiled ring 200 may be sutured around the circumference of a fenestration, threaded through a fabric tunnel surrounding a fenestration, and/or adhered to the circumference of a fenestration by an adhesive. A first end 201a of the coiled ring 200 can be attached to a control wire. The other, second end 201b of the coiled ring 200 can be attached/fixed to the stent graft itself. In various embodiments, the second end 201b of the coiled ring can be permanently fixed to the stent graft. In various embodiments, the second end 201b of the coiled ring can be removably attached to the stent graft. In various embodiments, the second end 201b of the coiled ring can be fixed to the stent graft by adhesive bonding, suturing, stapling, etc. In various embodiments, the first end 201a of the coiled ring can be permanently fixed to the control wire. In various embodiments, the first end 201a of the coiled ring can be removably attached to the control wire. Although the exemplary embodiment depicts a 5 mm diameter, alternative sizes are within the scope of the disclosure. For example, and without limitation, the diameter of the fenestration can be adjustable between 5 mm and 10 mm. Intermediate sizes such as 6 mm, 7 mm, 8 mm, or 9 mm may also be selected.

In operation, a user (i.e., a clinician or surgeon) may apply tension on the control wire attached to the first end 201a of the coiled ring 200. The user can do so by pulling on the control wire, e.g. in the direction “F” shown in FIG. 10, which consequently pulls the first end 201a of the coiled ring as well. Doing so effectively enlarges the diameter of the ring 200, and thereby applies a radially expansive force on the fenestration orifice, thereby increasing its diameter. Conversely, relieving the load “F” on the wire 201a allows the orifice to contract back to its nominal diameter. This design enables precise control over the orifice size, which is particularly useful for accommodating a bridging stent. This configuration enhances the adaptability and deployment efficiency of the stent-graft system.

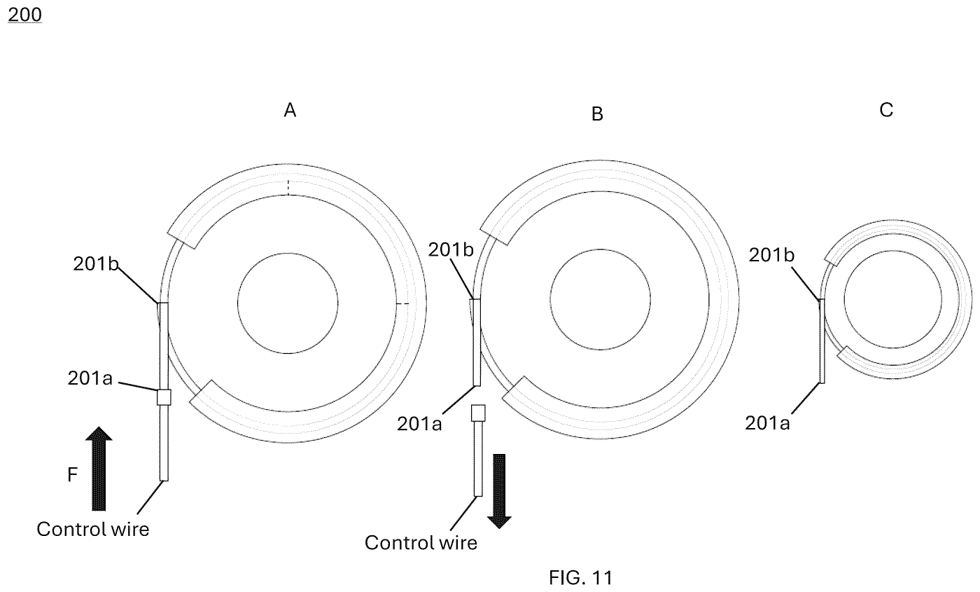

In various embodiments, the force on the coiled ring 200 results in a temporary deformation of the ring, meaning that the ring 200 can be iteratively expanded and compressed to simultaneously enlarge or reduce the diameter of the fenestration. This allows a physician to dynamically and repeatedly change the fenestration size to get a “feel” for what exact diameter is best for the particular bridging stent to be deployed through that fenestration. Once the desired diameter is obtained, the control wire can be decoupled from the ring 201a, as shown in FIG. 11.

In various embodiments, the coiled ring 200 is flexible enough such that tension can be applied to the control wire attached to the first end 201a of the coiled ring by hand. In various embodiments, the coiled ring 200 is rigid such that tension must be applied to the control wire attached to the first end 201a of the coiled ring by assistance with an instrument such as pliers.

In various embodiments, the coiled ring 200 has a resilient bias to return to an original size once a force from the control wire is removed, and can be made from a variety of suitable materials, e.g., a NiTi alloy. Nitinol alloys are favorable for use in sent grafts are they are generally well tolerated by the human body and tolerate repeated cycles of stress and strain well. In various embodiments, the coiled ring 200 may be formed from a copper-based alloy. In various embodiments, the coiled ring 200 may be formed from an iron-based alloy. In various embodiments, the coiled ring 200 may be formed from stainless steel. In various embodiments, the coiled ring 200 may be formed from a polymer or plastic. Additionally, the coiled ring 200 can be at least partially contained within a sheath to inhibit or prohibit damaging the fenestration ring/orifice. In such embodiments, the first end 201a can be exposed exterior of the sheath for engagement with the control wire, and the second end 201b can likewise be exposed exterior of the sheath for coupling to the graft.

FIG. 11 is a flow diagram illustrating a series of steps involved in adjusting the diameter of a fenestration with an exemplary coiled ring and control wire, according to various embodiments of the present disclosure. In step A, a pre-loading force “F” is applied to control wire, attached to the first end of the coiled ring 201a that increases the ring 200 diameter (e.g. from approximately 5 mm to approximately 10 mm). In step B, when the operator is ready to reduce the fenestration diameter, the control wire can be disconnected to relieve the expansion force “F”. In step C, the fenestration coil returns to it nominal diameter, e.g. 5 mm in the exemplary embodiment. In some embodiments, the Ring 600 is removable from the graft.

In various embodiments, the force required to pre-load the control wire in step A can be accomplished by hand. In various embodiments, the force required to pre-load the control wire in step A can be accomplished with the assistance of a tool, such as pliers.

In various embodiments, the control wire can be removed once the coiled ring 200 is adjusted to the desired size. In various embodiments, the control wire can be left attached to the coiled ring 200 after it is adjusted to the desired size.

### 3D Mesh Adjustable Fenestration

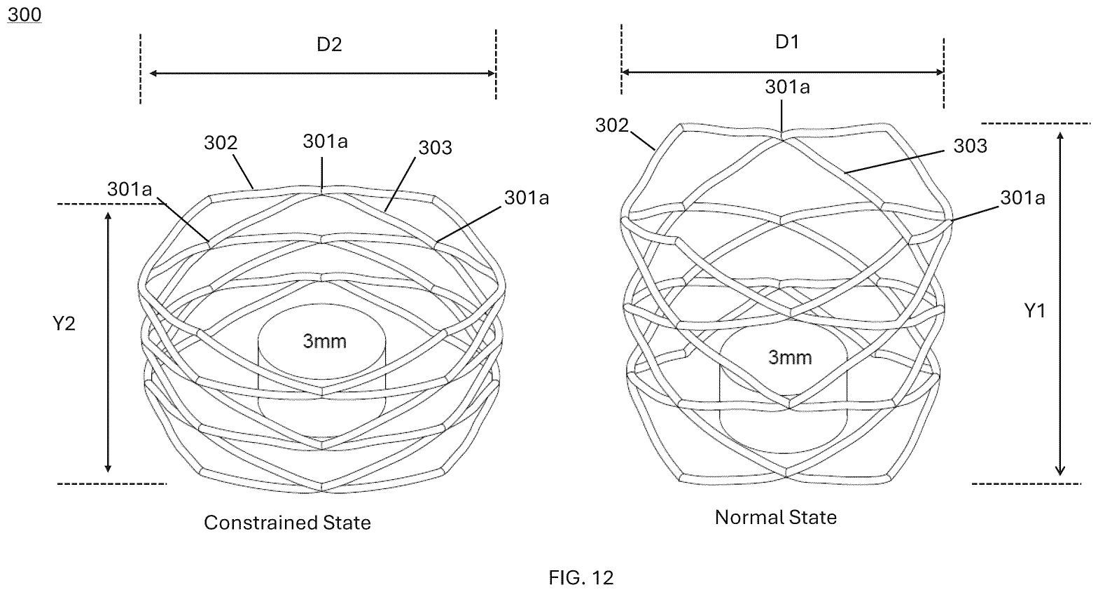

In various embodiments, the size of the fenestrations leading to the coronary ostium may be adjusted by a controllable orifice mechanism configured as 3D mesh structure (depicted as a stent in the exemplary embodiment of FIG. 12) that adjusts its diameter—again, or orifice width if non-circular—based on the vertical displacement of the structure.

FIG. 12 is an illustration of a perspective view of an exemplary 3D mesh structure 300 for adjusting the size of a fenestration, according to various embodiments of the present disclosure. The mesh structure 300 includes a series of wire stents crossed over one another at multiple points 301a to form a 3D cylindrical structure. In various embodiments, the mesh structure may be composed of various stents which overlap with each other at at least two points. The points at which two stents (302, 303) meet 301a may be fused such that as a tensional force is applied on one stent causing it to move, the connected stents may do the same. In various embodiments, the points at which two stents meet may be connected by a ring or band.

In various embodiments, each stent 302, 303 may include at two peaks and valleys. In various embodiments two consecutive stents 302, 303 may meet at the valleys of each stent. In various embodiments two consecutive stents 302, 303 may meet at the peaks of each stent. In various embodiments two consecutive stents 302, 303 may meet at the peak of one stent and the valley of the other.

In various embodiments, each stent can be made of any flexible and resilient metallic material, as known by those of ordinary skill in the art.

In various embodiments, each stent can be composed of a NiTi alloy. Nitinol alloys are favorable for use in sent grafts are they are generally well tolerated by the human body and tolerate repeated cycles of stress and strain well. In various embodiments, the each stent may be formed from a copper-based alloy. In various embodiments, the each stent may be formed from an iron-based alloy. In various embodiments, each stent may be formed from stainless steel. In various embodiments, each stent may be formed from a polymer or plastic.

In operation, the mesh structure 300 remains in a normal or default “closed” state as shown on the right side of FIG. 12, with a smaller diameter “D1” or orifice opening, and a greater height “Y1” (as compared to the constrained state). However, when the assembly is shortened (e.g., through compression) as depicted on the left side of FIG. 12, the diameter expands to “D2” while the height decreases to height “Y2”. In some embodiments, the amount of change in diameter size corresponds 1:1 to the vertical displacement of the structure.

In other words, as the stent 300 is compressed (vertically as shown in FIG. 12), its diameter increases (horizontally as shown in FIG. 12), and vice versa. This feature allows for precise control over the fenestration orifice size, making it especially useful for accommodating bridging stents. In some embodiments, the angles of the struts can be configured such that the amount of diameter expansion is greater than the amount of height constriction (e.g. 2:1). In some embodiments, the angles of the struts can be configured such that the amount of diameter expansion is less than the amount of height constriction (e.g. 0.5:1). For purposes of illustration and not limitation, the controllable orifice mechanism 300 can adjust the orifice diameter from approximately 10 mm to approximately 5 mm.

This fenestration orifice control mechanism 300 provides flexibility and mobility of the fenestration ring while being off the main body of the graft, and also presents a clear visual (e.g. can be formed of radiopaque material) marker of the exit of the fenestration that aids in cannulation. In various embodiments, the control mechanism 300 may be flexible enough to be compressed or expanded by the physician's hand, but rigid enough such that the structure does not change in diameter once deployed within a patient's body.

### Flexible Struts Adjustable Fenestration

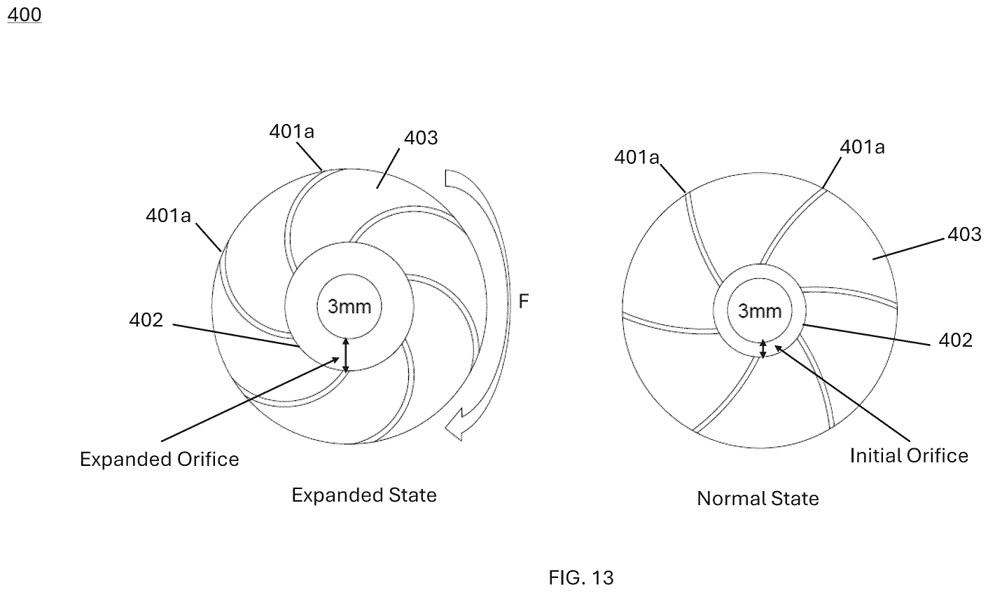

FIG. 13 is a front view of another controllable orifice mechanism configured in this exemplary embodiment as a wire strut fenestration adjustment mechanism 400, in accordance with various embodiments of the present disclosure. As discussed above, a stent graft may include multiple fenestrations 402 to support blood flow to branching vasculature (e.g., the coronary ostium). A wire strut assisted fenestration adjustment mechanism 400 can include a flexible or elastic fabric 403 partially or completely surrounding the fenestration site. A series of wire struts 401a may surround the fenestration 402, one end at the circumference of the fenestration 402 and extending radially outwards. Each, or select, wire struts 401a can be formed with a non-linear shape (e.g. arcuate) and may be composed of a flexible metal such as, but not limited to, NiTi. In some embodiments, each wire strut 401a has an identical shape (e.g. same contoured radius of curvature) so that the expansion force is equally distributed about the circumference of the fenestration.

In various embodiments, the wire struts 401a may be sutured to the flexible material 403 surrounding a fenestration. In various embodiments, the wire struts 401a may be attached to the flexible fabric 403 by an adhesive. In various embodiments, a pocket of fabric may be formed around a fenestration and each wire strut 401a may be sutured or glued to the fabric inside the pocket.

In various embodiments, a fenestration may be surrounded by at least three wire struts 401a. In various embodiments, a fenestration may be surrounded by at least four wire struts 401a. In various embodiments, a fenestration may be surrounded by at least five wire struts 401a. In various embodiments, a fenestration may be surrounded by at least six wire struts 401a. In various embodiments, a fenestration may be surrounded by more than six wire struts 401a.

One of ordinary skill in the art will recognize that the more wire struts 401a that surround a fenestration, the more force which will be required to deform the set of struts. Depending on a patient's needs, more or fewer struts may be added. For instance, in situations where flexibility of the fenestrations of a stent graft is required, fewer wire struts 401a can be added around the fenestration. Conversely, in situations where less flexibility of the fenestrations of a stent graft is required, more wire struts 401a can be added around the fenestration.

In various embodiments, the wire struts 401a may be removable and attachable from the elastic fabric of the stent graft, such that a user may customize the stent according to a patient's needs. In various embodiments, the wire struts 401a may not be removable from the stent graft.

In operation, the wire strut fenestration adjustment system 400 may exist in a default or normally closed state, when no force is applied to the system (as shown on the right of FIG. 13). In other words, the system may support a fenestration 402 with a smaller diameter in a resting state. When radial force is applied (as shown by arrow “F” in FIG. 13) to the circumference of the mechanism 400, the struts 401a may deform, e.g., by increasing the radius of curvature (or imparting a radius of curvature if linear struts are employed).

The deformation of the struts stretches the elastic/flexible fabric surrounding the fenestration 402 and accordingly stretches/increases the diameter of the fenestration (as labeled on the left side of FIG. 13).

In various embodiments, when the radial force is removed from the wire struts 401a of the fenestration adjustment mechanism 400, the mechanism is biased to resiliently return to the normally closed state, where the diameter of the fenestration 402 is constricted.

In various embodiments, the force required to deform the wire struts 401a of the fenestration 402 can be applied by hand. In various embodiments, the force required to deform the wire struts 401a of the fenestration 402 can be applied by a tool, such as a clamp.

In various embodiments, the force required to deform the wire struts 401a of the fenestration adjustment mechanism 400 can be applied to the outer perimeter formed by the wire struts (e.g., a squeezing force). In various embodiments, the force required to deform the wire struts 401a of the fenestration adjustment mechanism can be applied to the inner perimeter formed by the wire struts (e.g., a pushing force by a bridging stent).

In various embodiments, the diameter of the fenestration 402 can be adjusted prior to insertion into a patient's vasculature. In various embodiments, the diameter of the fenestration 402 can be adjusted after insertion into a patient's vasculature.

For purposes of illustration and not limitation, the controllable orifice mechanism 400 can adjust the orifice diameter from approximately 10 mm to approximately 5 mm.

### Lock Stent Valve Attachment Method

A stent graft 100, as described above in connection with FIGS. 3-5, can include a portion for attachment of a valve (for strengthening or replacement of the native aortic valve). Various methods for attaching a prosthetic valve to a stent graft may be used in practice.

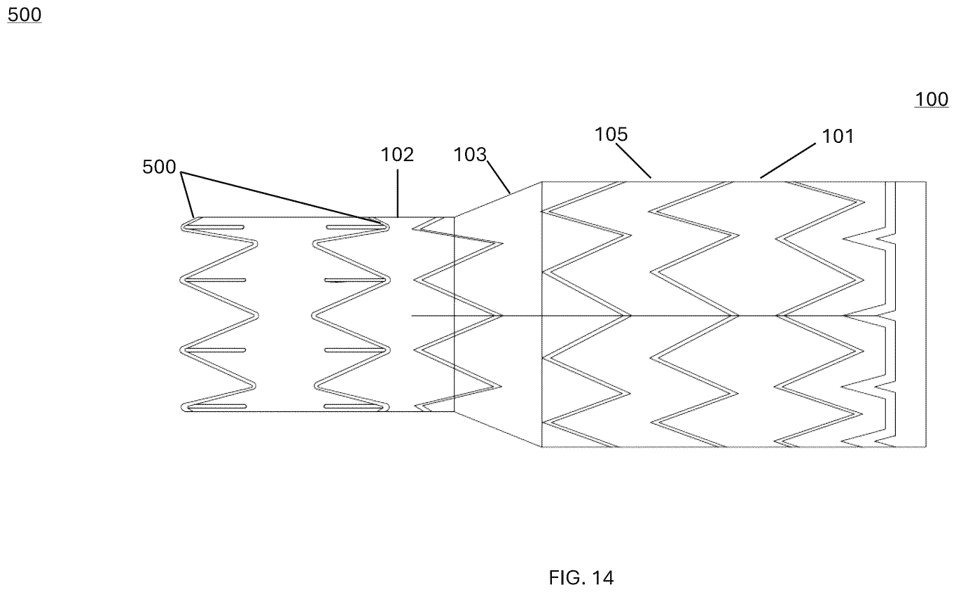

FIG. 14 is a side view of an exemplary valve and stent graft attachment by a series of locking stents 500, in accordance with various embodiments of the present disclosure. As depicted in FIG. 14, the stent graft can include a first portion 101 and second portion 102, as discussed in relation to FIG. 1, 2A-D. A valve can be inserted within the opening of the second portion 102 of the stent graft (and thus not visible from the exterior view in FIG. 14). In various embodiments, a valve can be positioned inside the opening of the second portion 102 of the stent graft, such that the valve and second portion are concentric. A portion of the valve may protrude from the opening of the second portion 102 of the stent graft. In various embodiments, the valve may be completely covered by the second portion 102 of the stent graft.

After the valve is positioned into the opening of the second portion 102 of the stent graft, a lock stent 500 can be added to the outer surface of the second portion 102 of the stent graft to secure the union between graft 102 and valve. The lock stent 500 can hold the valve in place within the second portion of the stent graft to prevent any unintentional movement (longitudinal and/or rotational) or slippage of the valve within a patient's vasculature.

In various embodiments, a single lock stent 500 can be used to hold the valve in place. In various embodiments, two or more lock stents 500 can be used to hold the valve in place.

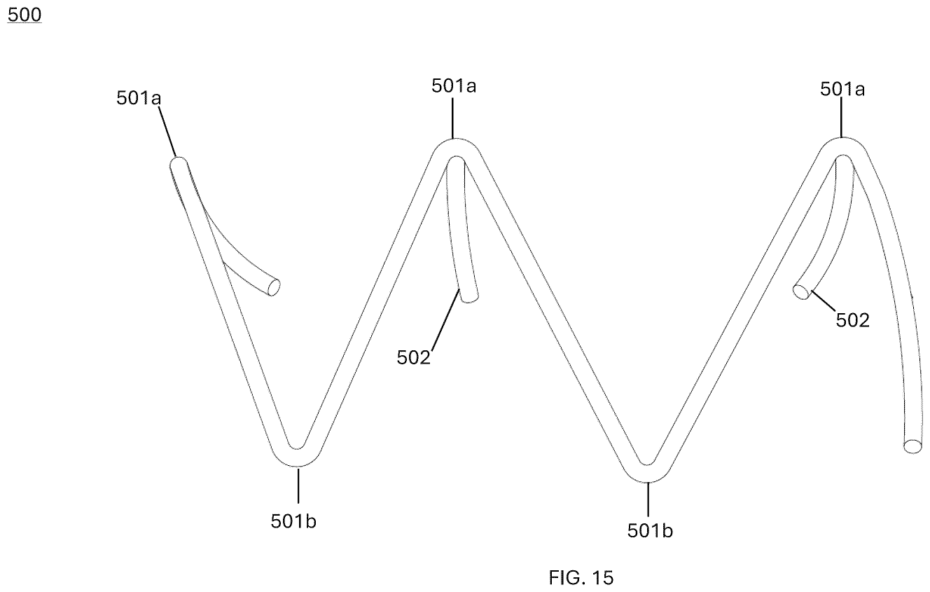

FIG. 15 illustrates an exemplary lock stent 500, in accordance with various embodiments of the present disclosure. An exemplary lock stent 500 may be circular in shape or may conform to the shape of an existing stent graft. The lock stent, made of a flexible or rigid metallic material, can include a series of adjacent peaks 501a and valleys 501b. An elongated portion 502 of the stent may extend downwards from each peak 501a and curve inwards towards the center of the stent. The elongated portion(s) 502 can serve as barbs that, in some embodiments, pierce the fabric of the graft 102 and valve (while avoiding the leaflet range of motion). Additionally or alternatively, the elongated portions 502 can interlock with struts of the stents within the graft 100 and elongated valve graft. In some embodiments, the elongated portion(s) do not pierce the graft 102, but apply a radially compressive force thereto to clamp the valve within the graft 102.

In various embodiments, the lock stent may include peaks 501a and valleys 501b that form sharp triangular points. In various embodiments, the peaks 501a and valleys 501b of a lock stent may form curved, non-sharp points.

In various embodiments, each peak 501a and valley of a lock stent 501b can form an angle between 0 -90 degrees. In various embodiments, the angle formed between each adjacent peak 501a and valley 501b of a lock stent can be the same. In various embodiments, the angle formed between each adjacent peak 501a and valley 501b can vary.

In various embodiments, the elongated portion 502 extending from each peak 501a of the lock stent 500 can extend through a portion of the height (or longitudinal span) of each peak. In various embodiments, the elongated portion 502 extending from each peak 501a of the lock stent can extend through the entire height of each peak. In various embodiments, the length of each adjacent elongated portion 502 can vary across the circumference of the lock stent. For example, and without limitation, a first elongated portion can possess a length L₁, while a second, adjacent elongated portion can possess a length, L₂. The third and fourth elongated portions can possess lengths of L₁ and L₂, respectively, and so on and so forth. In various embodiments, the length of each adjacent elongated portion 502 can remain constant across the circumference of the lock stent (in other words, the length of every elongated portion can be the same).

In various embodiments, every elongated portion 502 on the lock stent can be curved inwards by the same degree of curvature. In various embodiments, every elongated portion 502 on the lock stent can be curved inwards by the different degrees of curvature. For example, and without limitation, each adjacent elongated portion 502 may be curved at an alternating curvature. Continuing the example, a first elongated portion can possess a curvature C₁, while a second, adjacent elongated portion can possess a curvature, C₂. The third and fourth elongated portions can possess curvatures of C₁ and C₂, respectively, and so on and so forth.

In various embodiments, only the peaks 501a of the lock stent include a curved elongated portion 502. In various embodiments, only the valleys of the lock stent include a curved elongated portion 502. In various embodiments, both the peaks and valleys of the lock stent include a curved elongated portion 502.

In various embodiments where both the peaks 501a and valleys 501b of each lock stent include a curved elongated portion 502, each and every elongated portion 502 can extend in the same direction (i.e., proximally or distally with respect to the stent graft). In various embodiments, each of the elongated portions 502 located on peaks 501a of the lock stent can all extend in a first direction, while each of the elongated portions 502 on the valleys 501b of the lock stent can all extend in a second direction.

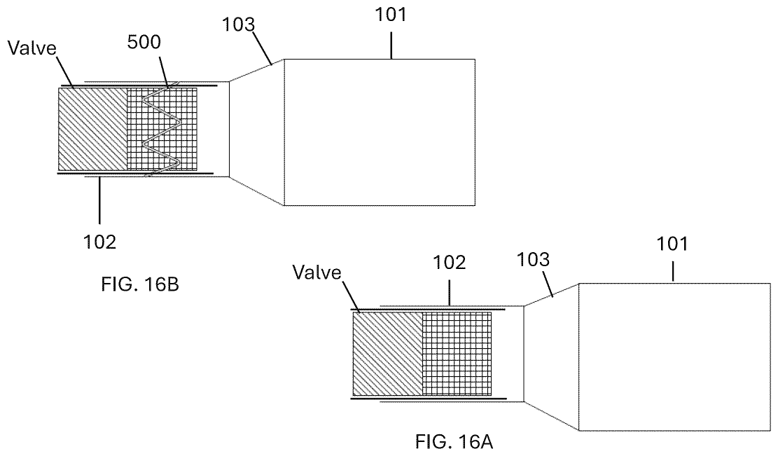

FIGS. 16A and 16B are exemplary stent grafts before and after (respectively) the addition of a locking stent to secure a valve thereto, in accordance with various embodiments of the present disclosure.

As described above, a valve can be positioned within the opening of the second portion 102 of a stent graft. A valve can be sized to establish an interference fit within the second portion 102 of stent graft. However, there can still be potential for slipping and movement of the valve within the stent graft 100. The positioning of one or more lock stents 500 along the surface of the second portion of the stent graft aids in anchoring the valve within the graft. Particularly, the curved elongated portions extending from the peaks and/or valleys of a lock stent 500 create anchoring points between the valve and stent graft. The ends of each curved elongated portion 502 can apply a radial inwards force that constrains a valve within the stent graft.

One of ordinary skill in the art will recognize that the greater number of lock stents 500 that are added to the stent graft and valve, the more constrained the valve will be and vice versa. As the number of lock stents 500 on the surface of the stent graft increases, as does the number of anchoring points formed by elongated portions 502.

In various embodiments, each lock stent 500 can be applied to the stent graft 100 and valve prior to implantation within a patient's vasculature. In various embodiments, lock stent 500 can be applied after the stent graft 100 and valve are deployed, thorough a second step where the lock stent 502 is tracked through a patient's aorta separately.

It should be recognized that the described lock stents can be used in conjunction with a variety of different stent graft, and not limited to those used to repair the aortic root. For example, and without limitation, lock stents can be used in stent grafts used in patients with abdominal aortic aneurysms (AAA) and can be used to constrain a bridging stent.

### Button/Magnet Valve Attachment Method

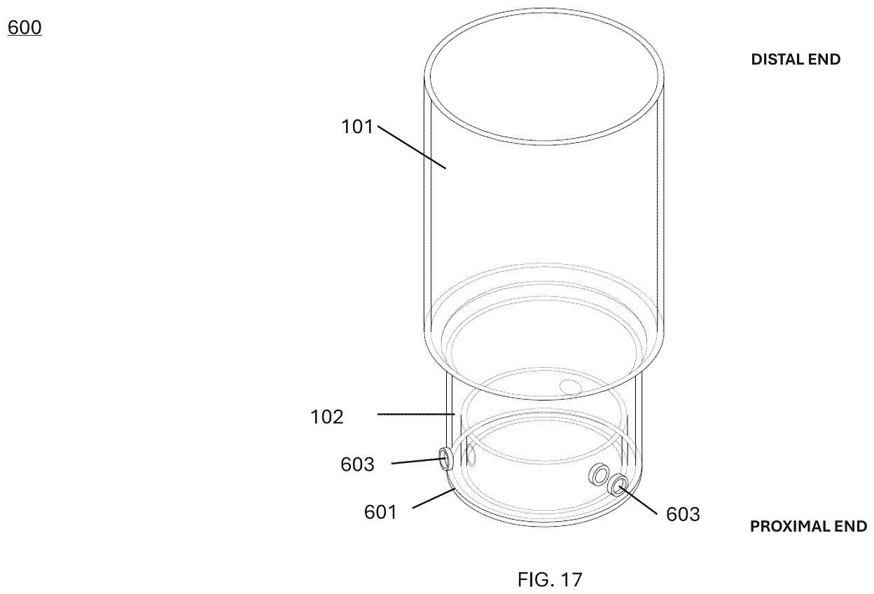

FIG. 17-27 are illustrations of another embodiment of a valve attachment to stent graft method which utilizes buttons and/or magnets in combination with a foldable or pleated portion at the proximal end of stent graft. This approach establishes a fast, secure and atraumatic method for joining a self-expanding valve member (with or without an elongated graft portion) to a stent graft (which can include fenestrations, bridging stents, etc. as described herein).

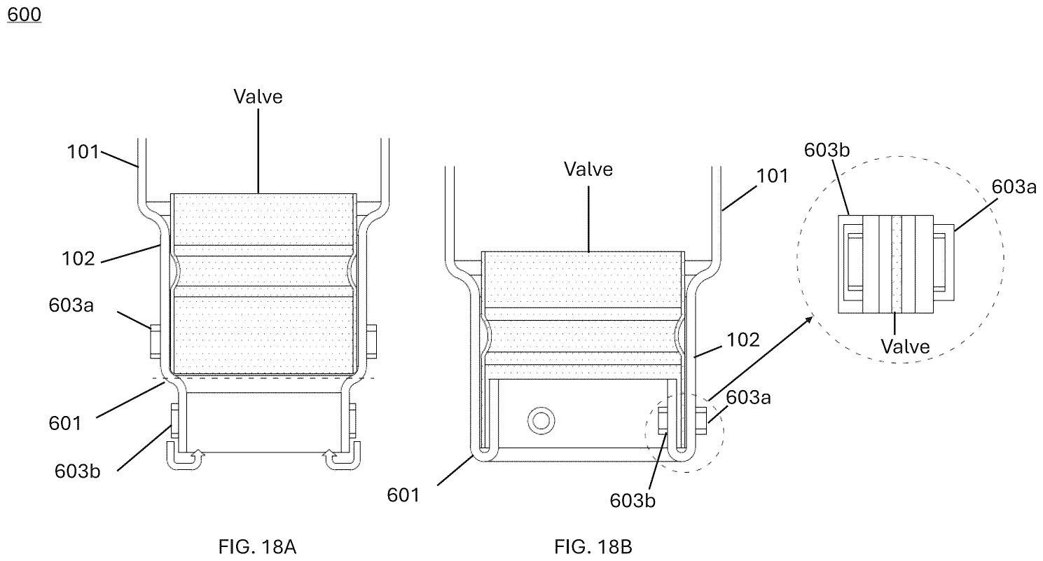

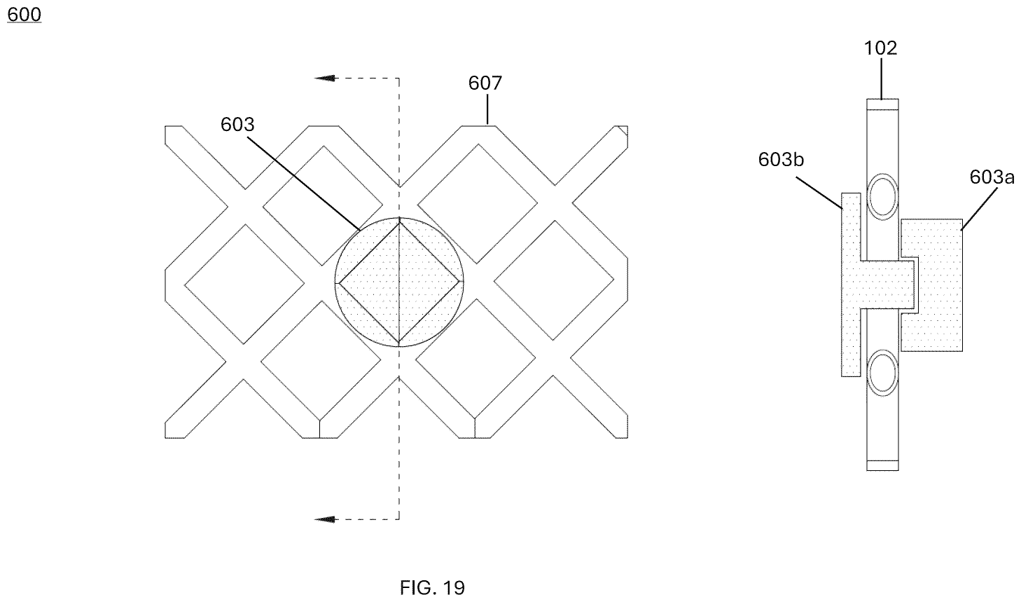

FIG. 17 is a transparent perspective view of an exemplary stent graft and valve attached by a button/magnet mechanism 600 that can be located on a folded/pleated portion of excess fabric at the proximal end of the graft. That is, a stent graft can include a pleat portion (indicated by reference line 601) on the second portion 102, allowing it to fold upwards into the inner portion of the stent graft. Once folded inwardly, the pleated section forms a recess or “pocket” to receive the proximal end of the valve (as shown in FIG. 18B). The outer surface of the second portion 102 of the stent graft may be fitted with one half of an attachment mechanism 603a. The folded portion of the second portion 102 can include the second half of an attachment mechanism 603b. When the pleated portion 601 of the stent graft is folded or flipped upwards the first half of the attachment mechanism 603a and the second half of the attachment mechanism 603a are mated together, securing the folded portion of the stent graft 601.

In various embodiments, the first half of an attachment mechanism 603a can be disposed on the outer surface of the second portion of a stent graft. The second half of an attachment mechanism 603b can be disposed on, what was initially an outer surface of the graft, but after being folded inwardly, is now on the interior side of the lumen. As shown in FIG. 18B, the attachment mechanisms 603a, b can be positioned on the stent graft such that they do not directly abut or engage the valve member, thereby avoiding damage to the valve. The formation of pleats in the foldable section allows for a smooth, wrinkle-free, surface on the interior of the lumen after flipping the foldable portion of excess fabric inwardly.

In various embodiments, a first attachment mechanism 603 and second attachment mechanism 603 can be disposed at diametrically opposing sides of the second portion of the stent graft. In various embodiments, a first attachment mechanism 603, second attachment mechanism 603, and third attachment mechanism 603 can be disposed at equally spaced positions of the second portion of the stent graft. In various embodiments, more than three attachment mechanisms 603 can be used to secure the pleat of the stent graft.

The attachment mechanisms provide a modular connection between the graft and valve, allowing various types of self-expanding valves to securely slot into the stent graft. This method utilizes the cavity or recess formed by the pleated/folded portion of the graft to provide a robust and adaptable attachment point. The amount of excess material forming the foldable portion can vary according to the size of the valve, with a larger foldable portion resulting in more longitudinal overlap between the valve and graft, and thus a more secure union. The foldable portion terminates proximal of the valve leaflets to avoid obstructing operation of the valve.

Additionally, in some embodiments the foldable portion does not extend around the entire circumference/perimeter of the graft, but instead is only present at select locations (e.g. diametrically opposed foldable tab portions). This design can be advantageous over fully circumferential attachment flaps as it maintains a clear passage and reduces potential flow disruptions while providing robust and secure valve fixation.

In various embodiments, the attachment mechanism 603 can include a button-type method. For example, and without limitation, a button-type attachment method can include a first male half and second female half which connect to one another by a snap fit mechanism. In various embodiments, the button-type attachment mechanism 603 may be attached to the stent graft by adhesive bonding, suturing, and/or any other suitable mechanism known to those of ordinary skill in the art. In other words, each half of the attachment mechanism (603a, 603b) can be attached to the stent graft by adhesive bonding, suturing, and/or any other suitable mechanism known to those of ordinary skill in the art.

In various embodiments, the attachment mechanism 603 can include a magnet-type method. For example, and without limitation, a magnet-type attachment method can include a first magnetic half and second magnetic half (603a, 603b) which connect to one another by when brought into proximity of one another. In various embodiments, the magnet-type attachment mechanism may be attached to the stent graft by adhesive bonding, suturing, and/or any other suitable mechanism known to those of ordinary skill in the art.

FIG. 18A and FIG. 18B depict a section view of the steps involved in securing a valve to a stent graft by a button/magnet style valve attachment mechanism taken along the z-axis.

FIG. 18A depicts a first step in a valve attachment method in accordance with various embodiments of the present disclosure. In operation, a valve can be fitted into the opening formed by the second portion of the stent graft. When the pleat portion 601 of the stent graft is folded upwards, the valve can sit such that the distal end of the valve is flush with the pleat 601 formed in the second portion 102 of the stent graft. In other words, when the pleat 601 is folded upwards, a pocket of space is formed between the second portion 102 of the stent graft and the pleat 601, which a valve can sit within. In various embodiments, the outer diameter of the valve can be approximately equal to the inner diameter of the second portion 102 of the stent graft, which has been slightly reduced due to the inwardly folded pleat portion 601, such that the valve fits snuggly into the stent graft.

In various embodiments, the valve can extend along the length of the second portion 102 of the stent graft. In various embodiments, the length of the valve may equal the length of the second portion 102 of the stent graft. In various embodiments, the valve can exceed the length of the second portion 102 of the stent graft and project distally outside or beyond the graft; and/or extend into the first portion 101 of the stent graft. In various embodiments, the valve can be shorter than the length of the second portion 102 of the stent graft.

In various embodiments, the valve may be inserted into the opening of the second portion 102 of the stent graft by hand. In various embodiments, the valve may be inserted into the opening of the second portion 102 of the stent graft by a tool, such as pliers. In various embodiments, the valve may be inserted into the opening of the second portion 102 of the stent graft after the stent graft is placed within a patient's vasculature. In various embodiments, the valve may be inserted into the opening of the second portion 102 of the stent graft prior to the stent graft being placed within a patient's vasculature.

FIG. 18B depicts the second step in a valve attachment method in accordance with various embodiments of the present disclosure.

In operation, once the valve is inserted into the second portion 102 of the stent graft, the pleat 601 formed on the second portion 102 can be folded upwards. The material forming the pleat 601 of the stent graft may fit into the opening formed the valve, securing the valve within the stent graft. Once the valve is secured between the inner surface of the second portion 102 of the stent graft and the inner portion of the pleat 601, the first half and second half of the button/magnet attachment mechanism 603 can be joined to one another.

In various embodiments, the first half 603a and second half 603b of a button-type attachment mechanism can be joined to one another by applying force to the first and second half in opposing directions. In various embodiments, the first half 6031a and second half 603b of a button-type attachment mechanism can be joined to one another by bringing the two sides into proximity of one another.

As can be seen in FIG. 18B, when a valve is secured to the second portion 102 of the stent graft by the method described, the valve is “sandwiched” by the second portion 102 of the stent graft and the pleat 601 of the stent graft. In other words, the valve is surrounded on its outer surface by the outer surface of the second portion 102 of the stent graft. The inner surface of the valve is surrounded by the pleated, folding portion 601 of the second portion of the stent graft. A first half of the attachment mechanism 603a is disposed on the outer surface of the second portion of the stent graft. A second half of the attachment mechanism 603b is disposed on the pleated, folding portion 601 of the second portion of the stent graft.

When a button-type attachment mechanism 603 is used to secure the valve, a portion of the first half or second half of the attachment mechanism 603 can pierce the valve to join with the other half of the attachment mechanism. When a magnet-type attachment mechanism 603 is used to secure the valve, the valve can be held within the fold formed by the pleat 601 of the stent graft through a compressive force, and without piercing any fabric on the graft (or valve, if present).

In various embodiments, when the pleated portion 601 of the stent graft is folded upwards into the valve, the pleat 601 may extend only partially along the length of the valve. The partial extension of the pleat along the length of the valve can prevent the leaflets of the valve from being obstructed.

FIG. 19 is a close-up view of the positioning of the attachment mechanism, in accordance with various embodiments of the present disclosure.

An exemplary stent graft can include wire stents which form a lattice structure 607, as known in the art. A first half 603a and second half of an attachment mechanism 603b can be positioned within openings of the lattice 607 of a stent graft. Such a positioning can prevent the attachment mechanism from disturbing the structure and stability of the stent graft. Accordingly, the presence of the attachment mechanism 603 does not impact any expansion or compression of the strut members.

In various embodiments, the lattice structure of the stent graft may include any known lattice structure known to those of ordinary skill in the art.

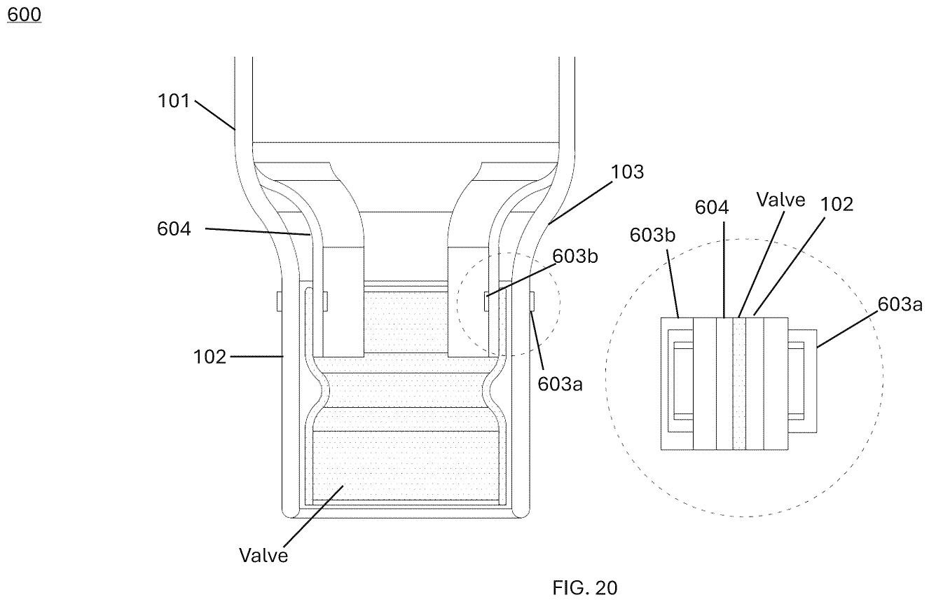

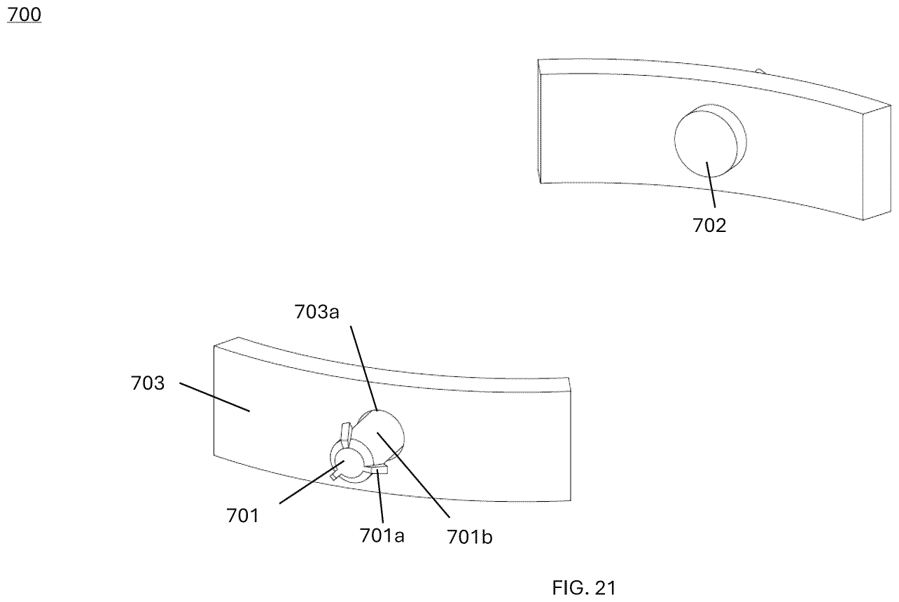

FIG. 20 is a sectional view of an alternative embodiment of a stent graft and valve attachment mechanism taken along the z-axis.

In the alternative embodiment depicted in FIG. 20, a stent graft may include at least one flap 604 disposed at a position within the inner surface of the stent graft and extending into the second portion 102 of the stent graft. The flap 604 can include one half of an attachment mechanism 603b as described above. The outer surface of the second portion of the stent graft can include the other half of the attachment mechanism 6003a at a position corresponding to the position of the half of the attachment mechanism 603b disposed on the flap 604.

Each flap 604 can include an elongated piece of fabric. In various embodiments, the flaps 604 can be rigid through reinforcement with metallic wiring. In various embodiments, the flaps 604 can be rigid due to the nature of the fabric. In various embodiments, the flaps 604 can be soft and flexible. In some embodiments, the attachment flap extends, from the wider portion 101 of the graft, and from the interior wall of the graft, and is received within the inner diameter of the valve, at the distal end of the valve.

In various embodiments, the flaps 604 may extend throughout the entire length of the second portion 102 of the stent graft. In various embodiments, the flaps 604 may extend through a portion of the second portion 102 of the stent graft.

In various embodiments, the flaps 604 can be rectangularly shaped pieces of fabric. In various embodiments, the flaps 604 can be triangularly shaped pieces of fabric, in order to reduce the amount of overlap between the flaps and the inner surface of the valve.

In various embodiments, the stent graft can include two flaps 604 disposed at diametrically opposing ends of the inner surface of the stent graft. In various embodiments, the stent graft can include three flaps 604 disposed at equally spaced apart positions of the inner surface of the stent graft. In various embodiments, the stent graft can include three flaps 604 disposed at unequally spaced apart positions of the inner surface of the stent graft. In various embodiments, the stent graft can include more than three flaps 604 disposed at equally spaced apart positions of the inner surface of the stent graft. In various embodiments, the stent graft can include more than three flaps 604 disposed at unequally spaced apart positions of the inner surface of the stent graft.

In various embodiments, the flaps 604 can be positioned along the inner surface of the transition portion 103 and can extend into the second portion 102 of the stent graft. In various embodiments, the flaps 604 can be positioned along the inner surface of the first portion 101 and can extend into the second portion 102 of the stent graft. In various embodiments, the flaps 604 can be positioned along the inner surface of the second portion 102 and can extend into the second portion 102 of the stent graft.

In various embodiments, the flaps 604 can extend only partially along the length of the valve. Doing so prevents the flaps 604 from obstructing the leaflets of the valve.

Depending on whether the leaflets of a valve are disposed on the proximal or distal end of the valve, the pleat attachment mechanism (described above) or the flap attachment mechanism can be used. One of ordinary skill in the art will appreciate the different ways in which the valve and attachment mechanism can be designed in order to enhance the function of the valve and prevent obstruction of its leaflets.

In operation, a valve may be inserted into the opening formed by the second portion 102 of the stent graft. The valve can be inserted such that the outer surface of the valve is surrounded by the second portion 102 of the stent graft and the flaps 604 of the stent graft sit within the opening of the valve and extend downwards.

When a button-type attachment mechanism 603 is used, opposing forces can be applied to both halves of the attachment mechanism. The opposing forces can cause the two halves to join together, securing the valve between the flaps 604 and stent graft. In various embodiments, the force required to join both halves of the attachment mechanism (603a, 603b) can be achieved by hand. In various embodiments, the force required to join both halves of the attachment mechanism (603a, 603b) can be achieved by the use of a tool, such as a clamp.

When a magnet-type attachment mechanism 603 is used, when the first half and second half of the attachment mechanism are brought into proximity of one another, the two halves join together. The attractive magnetic force between the two halves of the attachment mechanism secure the valve between the flaps 604 and stent graft. Depending on the needs of the patient and the preferences of the surgeon or clinician, the strength of the magnetic attraction between the two halves of the attachment mechanism (603a, 603b) can vary.

In various embodiments, a stent graft and valve may be packaged such that an attachment mechanism 603 is already disposed on the graft prior to use. In various embodiments, a valve attachment mechanism 604 may be disposed onto the graft by a user (i.e., clinician or surgeon) before or during the implantation of the stent graft in a patient's vasculature.

### Stamp and Anchor Attachment Mechanism

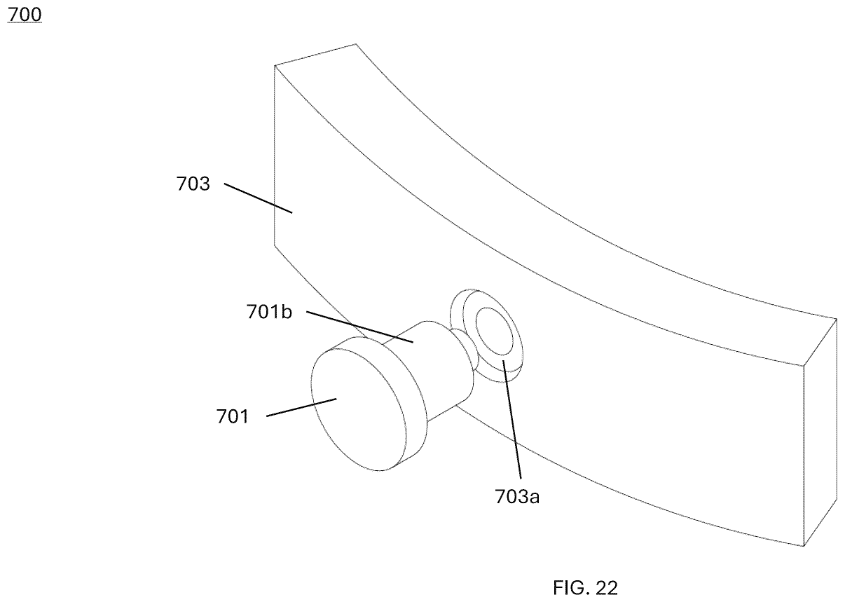

FIGS. 21 and 22 are perspective views of the front and back of another attachment mechanism for a securing a valve to a stent graft, in accordance with various embodiments of the present disclosure.

A valve attachment mechanism can include a first anchor 701, stamp 703, and second anchor 702. A male anchor 701 can include an elongated barbed member 701b, capable of piercing the fabric of the stent graft and the material of the valve. A stamp 703 can include an elongated material, conforming to the curvature/shape of the inner surface of the stent graft. The stamp can include an opening 703a extending through the front and back surface, corresponding to the shape of the elongated barb of the male anchor 701. At the end opposite to the barbed member 701b, the male anchor 701 can include a curved face that is flush with the curved surface of the stamp. A female anchor 702 can also include an elongated member with an opening extending through the member, corresponding to the shape of the barbed member 701b of the male anchor 701. The female anchor 702 can include a curved face on the side of the elongated member opposing the opening. The curved face can correspond to the curvature of the stamp 703, allowing it to lay flush with the surface of the stamp 703.

In various embodiments, the stamp 703 can possess a thickness and be composed of a rigid material that does not allow for bending or flexibility. In various embodiments, the stamp 703 can possess a thickness and can be composed of a flexible material, such as a fabric or plastic, that allows for bending and movement.

The stamp 703 can provide a level of structural stability to the stent graft and valve, preventing the assembly from sagging or being displaced. In various embodiments, the opening on the stamp 703a for accommodating the male and female anchors (701, 702) can be a simple circular opening, corresponding to the shape and size cross section of the male and female anchors (701, 702). In various embodiments, the opening on the stamp 703a can include an embossing, corresponding to the head of the male or female anchor (701, 702), such that the anchors sit flush with the surface of the stamp 703.

In various embodiments, the opening of the stamp 703a can be circularly shaped to correspond to the cross section of the male and female anchors (701, 702). In various embodiments, the opening of the stamp 703a can be shaped as a square to correspond to the cross section of the male and female anchors (701, 702). In various embodiments, the opening of the stamp 703a can be shaped as a triangle to correspond to the cross section of the male and female anchors (701, 702). In various embodiments, the opening of the stamp 703a can be shaped in any suitable manner to correspond to the cross section of the male and female anchors (701, 702).

In various embodiments, the curved face of the male anchor 701 can include a series of prongs 701a which extend radially out of the curved face and grip the surface they rest on. In various embodiments, the series of prongs 701a can be pointed to pierce and grip the surface on which they rest on. In various embodiments, the series of prongs 701a can be dull such that they merely sit on the surface on which they rest on. In some embodiments the prongs 701a serve as barbs which penetrate the stent graft fabric and the fixation tool subsequently deforms the anchor (e.g. the barbs 701a) thereby preventing its removal from the assembly, and forming a permanent lock between the valve and graft.

In various embodiments, the elongated members 701b of the male anchor 701 and female anchor 702 may be the same length. In various embodiments, the elongated members 701b of the male anchor 701 and female anchor 702 may correspond to the combined thickness of the stent graft and valve. In various embodiments, the elongated members of the male anchor 701 and female anchor 702 may be longer than the combined thickness of the stent graft and valve to allow for movement of the anchors, stent graft, and valve. In various embodiments, the elongated members of the male anchor 701 and female anchor 702 may be shorter than the combined thickness of the stent graft and valve to prevent any movement of the anchors, stent graft, and valve and promote tight compression between said components.

In various embodiments, the male anchor 701, stamp 703, and female anchor 702 can be joined by a compressive force applied by hand. In various embodiments, the male anchor 701, stamp 703, and female anchor 702 can be joined by a compressive force applied by a fixation tool.

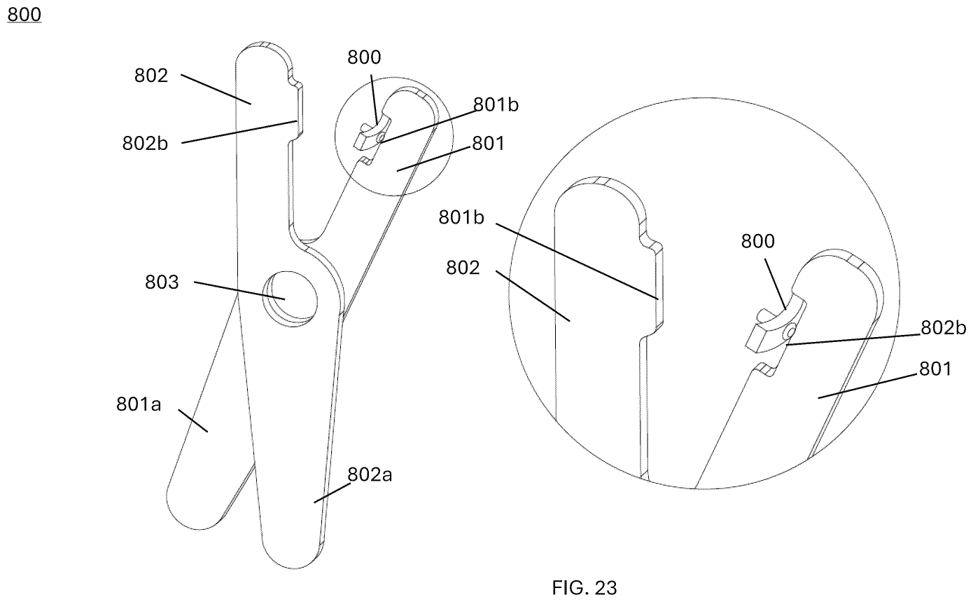

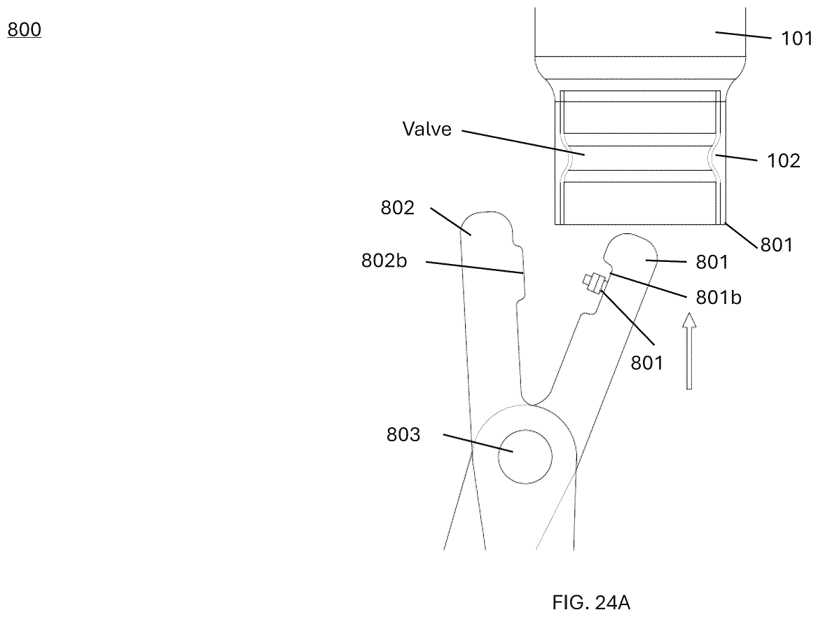

FIG. 23 is a perspective view of a male and female anchor fixation tool 800, in accordance with various embodiments of the present disclosure. A fixation tool 800 can include a first jaw/arm 801 and second jaw/arm 802 held together by a hinge or pin. The terms “jaw” and “arm” are used interchangeably herein and are understood to refer to the same component. The first jaw 801 and second jaw 802 can include a first 801a and second elongated portion 802a, respectively. Each of the elongated portions form a first and second handle for a user to grip during use.

The first jaw 801 of the fixation tool can include a cut-out 801b, where the male anchor or female anchor can be held. In various embodiments, the cut-out 801b can include a magnet for holding the male anchor or female anchor in place. In various embodiments, the cut-out 801b can include a set of prongs for holding the male anchor or female anchor in place. In various embodiments, the cut-out 801b can include a mild adhesive for holding the male anchor or female anchor in place.

The second jaw 802 can include an extrusion 802b at a location corresponding to the cut-out 801b on the first jaw. In various embodiments, the width of the extrusion 802b can match the width of the jaws of the fixation tool. In various embodiments, the width of the extrusion 802b can be smaller than the width of the jaws of the fixation tool. In various embodiments, the width of the extrusion 802b can be greater than the width of the jaws of the fixation tool. In various embodiments, the surface of the extrusion 802b can be covered by a foam or cushion-like material to avoid any damage to the anchors during a fixation process.

In operation, the stamp and anchor assembly 700 can be loaded into the cut-out of the first jaw 801 of the fixation tool. The male anchor and female anchor can be joined together loosely when affixed to the first jaw 801 of the fixation tool. Upon applying pressure to the first and second handle of the fixation tool (801a, 802a), the first jaw and second jaw (801, 802) are brought together to squeeze the anchor and stamp assembly 701. The extruded portion of the second jaw 802b applies a pressure onto the stamp and anchor assembly 701, locking the male anchor and female anchor together.

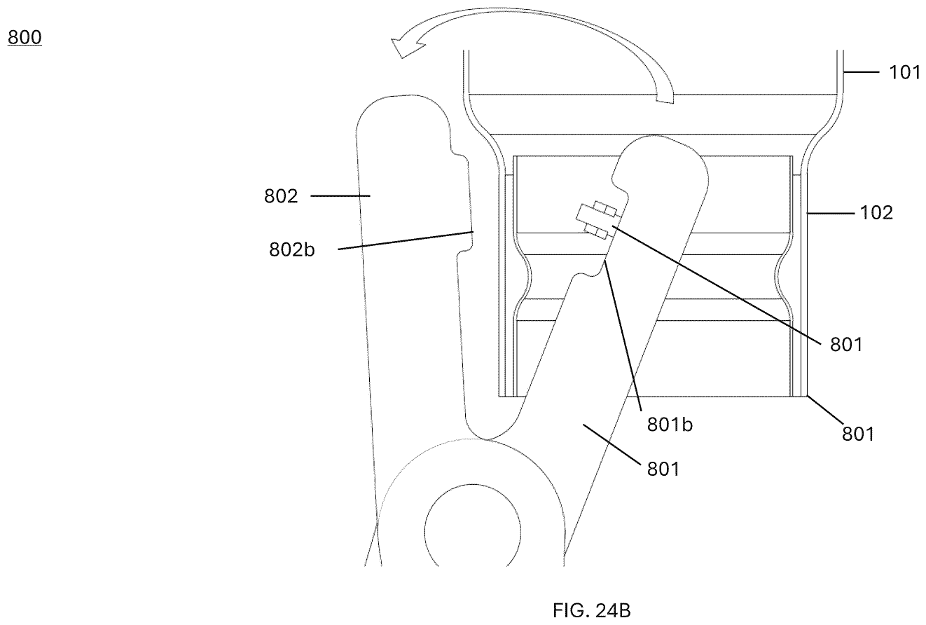

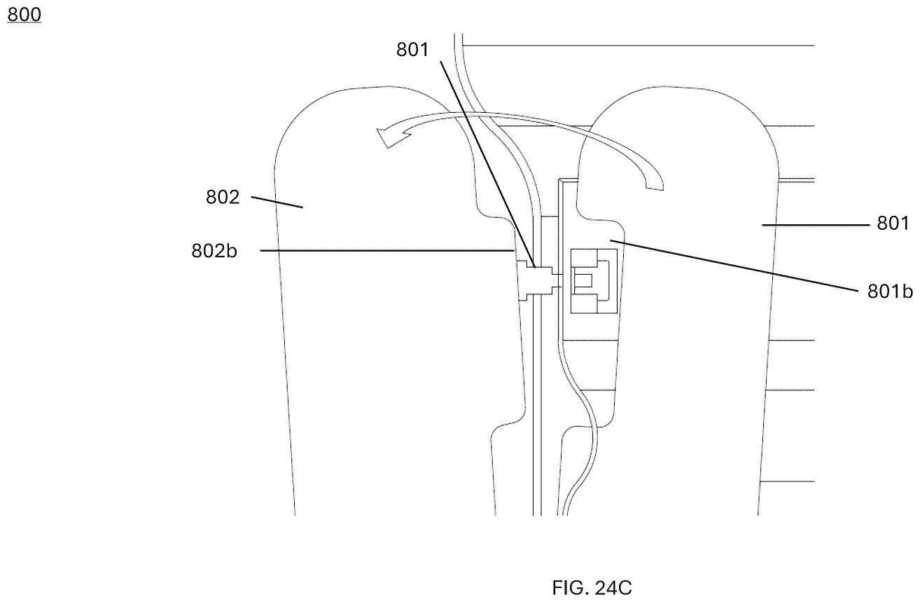

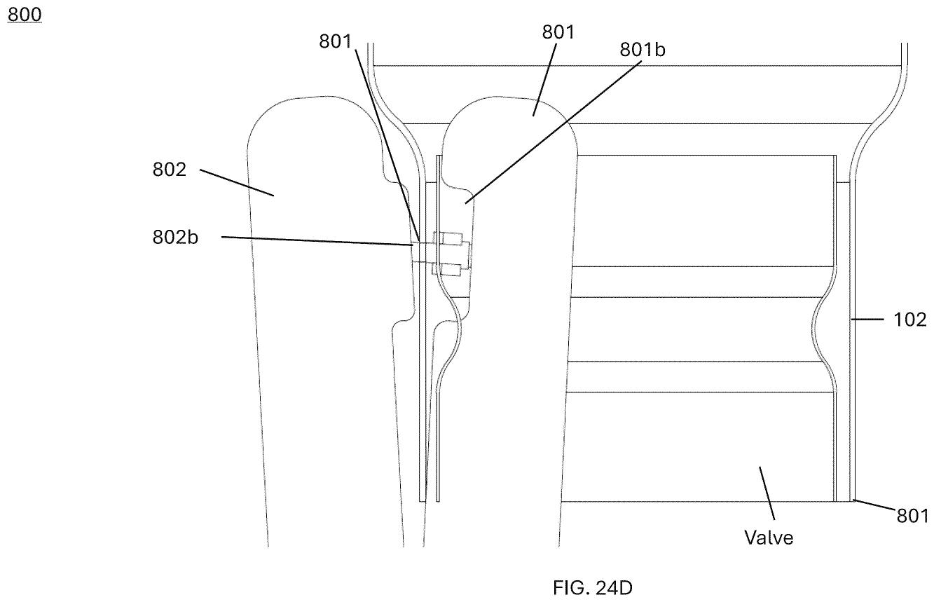

FIG. 24A-D depicts the series of steps involved in attaching a valve to a stent graft with an anchor and stamp assembly via a fixation tool.

FIG. 24A shows that in a first step the anchor and stamp assembly 700 can be loaded into the fixation device 800. Once the valve is inserted into the opening formed by the second portion of the stent graft, the first and second jaw (801a, 802a) of the fixation tool can be arranged around the valve and stent.

FIGS. 24B and 24C shows that in a second step the first jaw 801a can be brought into contact with the second jaw 802a. This can be accomplished by squeezing the first and second handle together (801a, 802a) in order to pierce the valve and stent graft with the male anchor and fix the assembly to the valve and graft. In various embodiments, the male anchor can include a sharp barb on its curved face (described above) which allows the assembly to pierce the valve and stent graft. In various embodiments, the female anchor and stamp, and male anchor may be mounted on the first jaw 801a and second jaw 802a respectively. Thus, when a squeezing force is applied to the jaws of the fixation tool, the male anchor may pierce the stent graft and valve to join with the female anchor.

FIG. 24D shows the third step where the anchor and stamp assembly is fully fixed to the stent graft and valve. When a squeezing pressure is released from the handles (801a, 802a) of the fixation tool, the tool may disengage with the male anchor and female anchor.

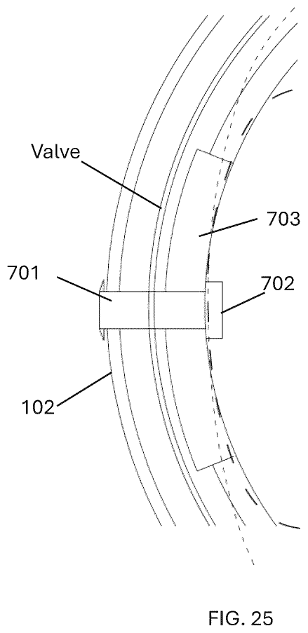

FIG. 25 is a top sectional view of an installed anchor and stamp assembly, in accordance with various embodiments of the present disclosure.

As can be seen in FIG. 25, when the stamp and anchor assembly is installed, the stent graft and valve are positioned between the male 701 and female anchor 702. The stamp 703 can be seen to conform to the curvature of the stent graft and valve.

In various embodiments, the free ends of the male anchor 701 and female anchor 702 may be flat. In various embodiments, the free ends of the male anchor 701 and female anchor 702 may be curved to conform to the shape of the stent graft and valve. In various embodiments, the free ends/head of the male anchor 701 and female anchor 702 may be chamfered.

In various embodiments, the free end of the male anchor 701 may rest on the outer surface of the stent graft when the stamp and anchor assembly is installed. In various embodiments, the stamp and anchor assembly can be installed such that the free end/head of the female anchor 702 may rest on the outer surface of the stent graft.

In embodiments where a valve and any one of the attachment mechanisms of FIG. 14-25 are employed, the valve and/or attachment mechanism may completely surround the circumference of the second portion 102 of the stent graft and completely or almost completely extend across the length of the second portion 102 of the stent graft. Thus, it may be necessary to adjust the positions of the pair of fenestrations leading to the coronary arteries. In various embodiments, a pair of fenestrations may be disposed on the first portion 101, the distal end of the second portion 102, and/or the distal end of the transition portion 103 of the stent graft. By situating the fenestrations on the first portion 101, the second portion 102, and/or the transition portion 103 of the stent graft so that they do not interfere with the valve or its attachment mechanism, such that guidewires and/or bridging stents can be advanced to the coronary arteries through the graft without obstruction.

### Fabric Constraint Valve Attachment Method

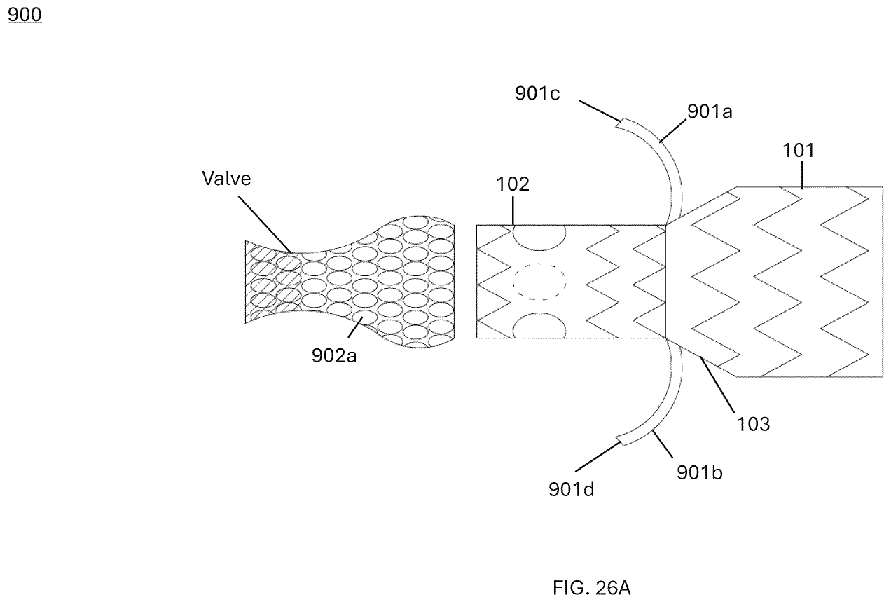

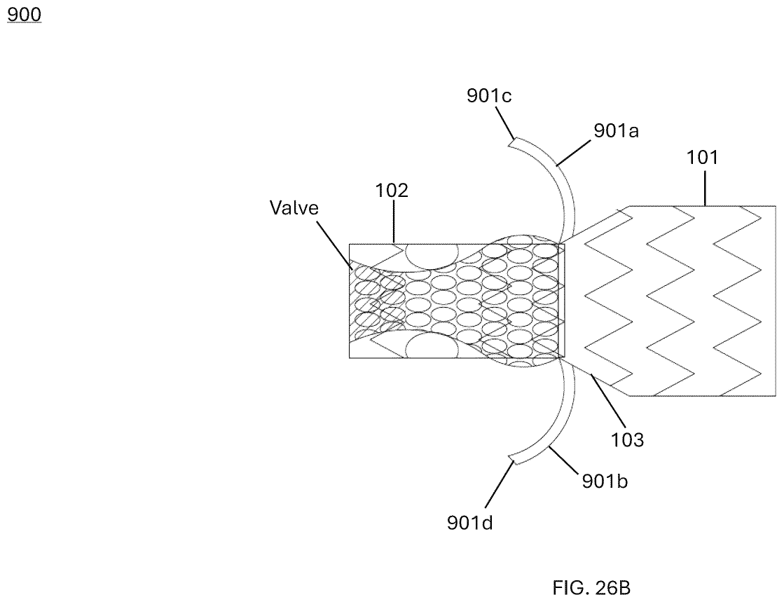

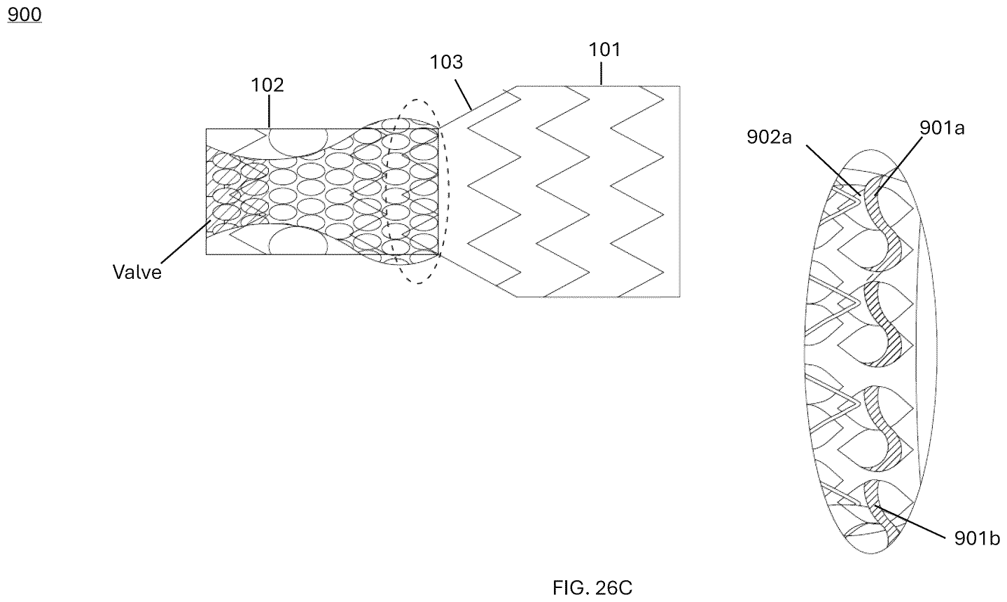

In various embodiments, a valve may be fixed to the stent graft by fabric constraints. FIG. 26A-C are illustrations of a side view of a fabric constraint attachment mechanism 900, in accordance with various embodiments of the present disclosure. In various embodiments, a stent graft 100 can be composed of a wire material formed in a grid-like pattern 902a. The second portion 102 of the stent graft can include a first and second fabric constraint (901a, 901b). Each of the first and second fabric constraint (901a, 901b) can be an elongated piece of fabric, wherein the first and second elongated constraint include a first end and second end thereby defining a length extending therebetween, with one end fixed to the stent graft and the other end free. In various embodiments, at least one of the first and second fabric constraint (901a, 901b) are fixed to an inner surface of the expandable graft. In various embodiments, at least one of the first and second fabric constraint (901a, 901b) are fixed to an exterior surface of the expandable graft. In various embodiments, the each of the first and second fabric constraint (901a, 901b) can match the length of the circumference of the stent graft. In various embodiments, the each of the first and second fabric constraint (901a, 901b) can be longer than the circumference of the stent graft. In various embodiments, each of the first and second fabric constraint (901a, 901b) can be shorter than the circumference of the stent graft. In various embodiments, the first and second fabric constraint (901a, 901b) can be different lengths. In various embodiments, the first and second fabric constraint (901a, 901b) can be the same length.

In various embodiments, each of the first and second fabric constraint (901a, 901b) can be composed of a loose fabric material, free from any reinforcements. In various embodiments, each of the first and second fabric constraint (901a, 901b) can be composed of a fabric material reinforced with a wire lattice to provide some structural support to the constraints.

In various embodiments, the first and second fabric constraints (901a, 901b) can be disposed 180 degrees apart from each other on the stent graft. In various embodiments, the first and second fabric constraints (901a, 901b) can be disposed at positions less than 180 degrees apart on the stent graft.

In various embodiments, the first fabric constraint and second fabric constraint (901a, 901b) can be disposed at the distal end of the second portion of the stent graft. In various embodiments, the first fabric constraint and second fabric constraint (901a, 901b) can be disposed at any position along of the second portion of the stent graft.

As shown in FIG. 26A-C, the valve may also be composed of a material formed in a grid-like pattern. In operation, when the valve is inserted into the second portion 102 of the stent graft, the first and second fabric constraint (901a, 901b) can be woven through the openings of the grid 902a formed by the valve and/or graft. Once the fabric constraints (901a, 901b) are woven in between the grid structure of the valve and/or graft, the free ends (901c, 901d) can be fixed to one another.

In various embodiments, the free ends (901c, 901d) of the first fabric constraint and second fabric constraint (901a, 901b) can be sutured to one another. In various embodiments, the free ends of the first fabric constraint and second fabric constraint can be tied to one another. In various embodiments, the free ends (901c, 901d) of the first fabric constraint and second fabric constraint (901a, 901b) can be glued to one another. In various embodiments, the free ends (901c, 901d) of the first fabric constraint and second fabric constraint (901a, 901b) can be connected to one another by any suitable method known to one of ordinary skill in the art.

In various embodiments, the first and second fabric constraint (901a, 901b) can be woven in-between the grid-like structure 902a of only the valve in order to hold it place within the stent graft. In various embodiments, the first and second fabric constraint (901a, 901b) can be woven in-between the grid-like structure 902a of the stent graft and valve for additional reinforcement and security.

In various embodiments, the valve can be inserted into the opening formed by the second portion 102 of the stent graft. In various embodiments, the valve can be inserted over the outer surface of the second portion 102 of the stent graft.

One of ordinary skill will recognize that using fabric constraints (901a, 901b) to secure a valve within a stent graft 100 allows for minimal contact between the constraints and the valve itself, preventing any obstruction of the leaflets. Furthermore, fabric constraints (901a, 901b) can be easily customizable. Prior to use a surgeon or clinician can cut the constraints (901a, 901b) to their desired size and length. During attachment of the constraints (901a, 901b) onto the stent graft, their placement can be chosen as desired or needed.

In various embodiments, more than two fabric constraints (901a, 901b) can be used to hold a valve within a stent graft 100. For example, and without limitation, a first and second fabric constraint (901a, 901b) can be placed at the proximal end of the second portion 102 of the stent graft 100. A third and fourth fabric constraint (not shown) can be positioned at the distal end of the second portion 102 of the stent graft 100. In various embodiments, additional fabric constraints can be positioned between the proximal and distal ends of the second portion 102 of the stent graft 100.

In embodiments where a valve and the attachment mechanisms of FIG. 26A-C are employed, the valve may completely surround the circumference of the second portion and completely or almost completely extend across the length of the second portion 102 of the stent graft. Thus, it may be necessary to adjust the positions of the pair of fenestrations leading to the coronary arteries. In various embodiments, a pair of fenestrations may be disposed on the first portion 101 or the transition portion 103 of the stent graft. Positioning the fenestrations on the first portion 101 or the transition portion 103 of the stent graft such that they do not interfere with the valve and attachment mechanism allows for guide wires and/or bridging stents to the coronary arteries to extend through the graft without interference.

In various embodiments, the valve may be formed with a frustoconical, or tapered, geometry such that the proximal end of the valve possesses an outer diameter that is intentionally smaller than the inner diameter of the second portion 102 of the stent-graft. This offset in diameters establishes a clearance or “open space,” between the exterior surface of the valve and the interior wall of the surrounding graft material. By positioning a pair of fenestrations on the graft wall at a position that coincides with the region of clearance/“open space”, a conduit through which guidewires may be advanced or withdrawn along the full length of the stent-graft without physical impingement by the valve structure is formed.

One of ordinary skill in the art will also recognize that the fabric constraint mechanism can be used to secure components other than valves to a stent graft. For example, and without limitation, fabric constraints can be used to secure bridging stents, hooks, barbs, and/or anchoring pins to a stent graft.

In accordance with an aspect of the disclosure, this technique secures the valve graft to the stent graft 100, but does not directly sew the two components together—thus reducing the probability of damage to the valve. Additionally, the fabric constraints can be used on a wide variety of valve structures and is not limited to one type of valve system. This allows the valve to maintain an open segment for profusion of the coronary arteries.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed:

1. A medical device comprising:
a first expandable graft having a proximal and a distal end defining a graft lumen extending therebetween;
the graft having a first portion defining a first diameter;
the graft having a second portion defining a second, different diameter;
a first tunnel lumen having a proximal end and a distal end defining a first tunnel lumen therebetween, the first tunnel lumen longitudinally aligned with the graft lumen;
a second tunnel lumen having a proximal end and a distal end defining a second tunnel lumen therebetween, the second tunnel lumen longitudinally aligned with the graft lumen;
wherein the proximal end of the first graft lumen is configured to couple with a valve, wherein the graft lumen trifurcates in a retrograde manner parallel with the first tunnel and second tunnel, the first tunnel configured to receive a first bridging stent and second tunnel configured to receive a second bridging stent.

2. The medical device of claim 1, wherein the first expandable graft includes a transition portion between the first portion and the second portion, wherein the transition section provides a gradual change in diameter from the first diameter to the second diameter.

3. The medical device of claim 2, further comprising a first fenestration and a second fenestration located on opposing sides of the transition portion, the first fenestration in fluid communication with the first tunnel lumen and the second fenestration in fluid communication with the second tunnel lumen.

4. The medical device of claim 3, wherein the angle between the first fenestration and second fenestration is approximately 100 degrees to approximately 140 degrees.

5. The medical device of claim 1, wherein the diameter at the distal ends of the first and second tunnel lumens is different than the diameter at the proximal ends thereof.

6. The medical device of claim 3, wherein the diameter of at least one of the first fenestration and second fenestration is adjustable.

7. The medical device of claim 3, further comprising a first angled opening proximal to the first fenestration and a second angled opening proximal to the second fenestration.

8. The medical device of claim 3, wherein at least one of the first fenestration and second fenestration is surrounded by a ring.

9. The medical device of claim 8, wherein a first end of the ring is removably coupled to a control wire and a second end of the ring is coupled to the graft.

10. The medical device of claim 9, wherein a radial load is applied to the ring of at least one of the first fenestration and second fenestration, thereby increasing its diameter, when tension is applied via the control wire.

11. The medical device of claim 3, wherein the diameter of at least one of the first fenestration and the second fenestration is adjustable from approximately 5 mm to approximately 10 mm.

12. The medical device of claim 3, wherein at least one of the first fenestration and the second fenestration is surrounded by a set of radially extending struts.

13. The medical device of claim 12, wherein when a radial load is applied to the struts, the struts deform to expand the diameter of the at least one fenestration.

14. The medical device of claim 12, wherein when a radial load is removed from the struts, the struts constrict the diameter of the at least one of the first fenestration and second fenestration.

15. The medical device of claim 3, wherein at least one of the first fenestration and second fenestration is surrounded by an elastic fabric to adjust the diameter of said fenestration.

16. The medical device of claim 1, wherein the valve is coupled to the second portion of the first graft by at least one lock stent.

17. The medical device of claim 16, wherein the lock stent comprises:
a tubular body composed of annular rings, each annular ring including a series of peaks and valleys;
an elongated portion extending longitudinally from each peak.

18. The medical device of claim 17, wherein each elongated portion of the lock stent is curved radially inward.

19. The medical device of claim 16, wherein a second lock stent is positioned spaced apart from a first lock stent such that each peak of the first lock stent is longitudinally aligned with each peak of the second lock stent wherein the elongated portions of the first and second lock stent extend along opposite directions from one another.

20. The medical device of claim 1, wherein a second graft is at least partially positioned within the first graft such that a fenestration formed in the second graft is at least partially aligned with a fenestration formed in the first graft, and wherein the second fenestration is surrounded by a radiopaque ring.

21. The medical device of claim 20, wherein the first fenestration and second fenestration are concentrically aligned.

22. The medical device of claim 20, wherein the first fenestration is surrounded by a radiopaque ring.

23. The medical device of claim 20, wherein a longitudinal length of the second graft is less than a longitudinal length of the first graft.

24. The medical device of claim 20, wherein a diameter of the fenestration formed in the second graft is less than a diameter of the fenestration formed in the first graft.

25. The medical device of claim 1, wherein the proximal end of the first portion includes a foldable pleat forming a cavity between an inner surface of the graft and the folding pleat.

26. The medical device of claim 25, wherein the cavity is configured to receive a portion of the valve.

27. The medical device of claim 25, wherein the pleat includes at least one locking mechanism.

28. The medical device of claim 27, wherein the at least one locking mechanism includes a magnet disposed on the foldable pleat and a mating magnet disposed on an exterior surface of the first graft.

29. The medical device of claim 27, wherein the at least one locking mechanism includes a male button disposed on the first graft and a female button disposed between adjacent struts of an annular stent of the valve, the male button received within the female button in a locked configuration.
